(19) United States
(12) Patent Application Publication
(10) Pub. No.: US 2026/0124216 A1
(43) Pub. Date: May 7, 2026

(54) METHOD FOR INCREASING POLLINATION IN CROPS SUSCEPTIBLE TO POLLINATION OF INVERTEBRATES OF THE APIDEAE FAMILIES

(71) Applicants: APIX BIOSCIENCES N.V., Wingene (BE); PHYTANT, Wingene (BE)

(72) Inventors: Thierry BOGAERT, Wingene (BE); Jan BOGAERT, Wingene (BE)

(73) Assignees: APIX BIOSCIENCES N.V., Wingene (BE); PHYTANT, Wingene (BE)

(21) Appl. No.: 18/939,792

(22) Filed: Nov. 7, 2024

(51) Int. Cl. A01K 67/368; A01G 9/14; A01G 13/24; A01K 47/06; A61K 31/198; A61K 31/202; A61K 31/352; A61K 31/575; A61K 45/06

(52) U.S. Cl. CPC A61K 31/575; A61K 31/198; A61K 31/202; A61K 31/352; A61K 45/06

(57) ABSTRACT

A method for increasing pollination, crop quality and/or crop yield and/or seed production in agricultural or horticultural crops susceptible of pollination through invertebrates of the Apidae families (bees), in particular honey bees (Apini), bumble bees (Bombini), or stingless honey bees (Meliponini), including providing agricultural, horticultural crops, or bee-yards where bees are placed in transit prior to or after pollination, providing one or more beehives within or in the immediate vicinity of the agricultural or horticultural crops to pollinate the agricultural or horticultural crops during the pollination period of the agricultural or horticultural crops or greenhouse crops or partially covered crops, in particular tunnel crops, and administering to the beehives a pollen supplement composition.

### FIELD OF THE INVENTION

The present invention relates to a method for increasing pollination, crop quality, crop yield and/or seed production in agricultural or horticultural crops susceptible of pollination through invertebrates of the Apidae families (bees), in particular honey bees (Apini), bumble bees (Bombini), or stingless honey bees (Meliponini).

### BACKGROUND

Honeybees, the principal managed pollinators, are essential for global agricultural economies, global food security and diversity. Worldwide, around 70% of crop plants accounting for 35% of production volume rely on pollination by insects to some degree (14) (15) (16) (17).

Few plants produce pollen that meet all nutritional requirements of honeybees. Many crops (including blueberries and sunflowers) produce pollen that are nutritionally inadequate for honeybees. Hence, bees require access to pollen from different floral sources (10).

At the end of the summer, longer lived winter bees are produced that hibernate together with the queen and raise the first generation of new worker bees in the spring. The colonies require well fed and long-lived winter bees (120 days) to successfully overwinter and set up the next generation.

Continuous brood production is required to ensure effective functioning of the colony (10). Inadequate or interrupted colony nutrition results in gaps in brood production, gaps in distinct age cohorts delivering a specific task in the hive (18) (19) and bees with reduced longevity, reduced flight capabilities, reduced behavior, and reduced homing and communication capabilities, each a strong impactor of colony growth. A pollen or nutrition gaps have season-long impacts on colony health and immunity, tolerance to stressors (pathogens, pesticides, weather) (20) (21) (22) (23) (24) and may lead to colony mortality (25) (26) (27) (28).

Many honeybee colonies are chronically starved due to a lack of a continuous supply of nutritionally adequate pollen from diverse flower sources. These shortages are driven by reduced availability of pesticide free forage areas, and reduced flower diversity due to monoculture agriculture and urbanization. This is exacerbated by climate change that produces unpredictable flowering patterns, untimely rain and cold events as well as extreme weather events (26) (29).

Overall, this malnutrition impairs the colonies' ability to withstand pathogens, pesticides, and abiotic stressors, and results in colonies that pollinate less efficiently, and the unsustainable rates of colony morbidity and mortality observed today in many countries. It is well established that well-fed bees and larger colonies are more effective in pollinating crops and results in higher crop yield and quality.

It is not likely that our landscapes will revert to the florally rich biodiverse landscapes honeybees require, and that agriculture will become less intensive. Feeding adequate pollen replacement to honeybees is potentially part of a long-term solution.

To mitigate insufficient and unpredictable pollen availability, many beekeepers today stock hives at low densities in distantly spaced bee-yards and relocate colonies over long distances to areas with natural pollen and nectar flow where they can impact native wildlife and plant biodiversity (18).

In the U.S., many professional beekeepers provide supplementary feeds to their colonies, often in the form of a dough-like protein supplement patty, from September onwards to prepare their colonies for winter and in the spring to boost colony growth in January ahead of almond pollination.

Field trials of currently available pollen patties during periods when there is pollen flow have not shown a utility in increasing honeybee performance.

Commercially available pollen supplement patties lack nutritional balance and cannot sustain colonies without access to natural pollen (30) (31).

The inventors showed this in the reference examples below with two leading and widely used commercial protein supplements for honeybees.

### SUMMARY

The present inventors surprisingly found that addressing dietary deficits in the pollen of a mono crop before, during, and/or after deployment with a pollen supplement composition increases the fitness of the pollinators deployed to the crops and thereby the pollination efficiency, crop quality, crop yield and/or seed production in agricultural or horticultural crops susceptible of pollination through invertebrates of the Apidae families (bees), in particular honey bees (Apini), bumble bees (Bombini), or stingless honey bees (Meliponini).

The artificial pollen supplement composition is particularly useful for honeybees to provide a complete pollen-replacing feed and can improve the performance of honeybees while deployed in commercial pollination in field crops and greenhouse crops and other stressful conditions such as storage in bee-yards.

Accordingly, a first aspect of the present invention is a method for increasing pollination, crop quality and/or crop yield and/or seed production in agricultural or horticultural crops susceptible of pollination through invertebrates of the Apidae families (bees), in particular honeybees (Apini), bumblebees (Bombini), or stingless honey bees (Meliponini), comprising:
providing agricultural, horticultural crops, or bee-yards where bees are placed in transit prior to or after pollination;
providing one or more beehives within or in the immediate vicinity of the agricultural or horticultural crops to pollinate the agricultural or horticultural crops during the pollination period of the agricultural or horticultural crops or greenhouse crops or partially covered crops, in particular tunnel crops;
administering to the beehives a pollen supplement composition
   2-3 months prior to entry and at entry
   during a part or the full pollination period of the agricultural or horticultural crops; and/or
   during transportation from one crop to another crop; and/or
   in the holding yards;
wherein the pollen supplement composition compensates for one or more dietary deficits in the amount or quality of the pollen of the agricultural or horticultural crops; and/or in the environment;
wherein the pollen supplement composition is free of pollen or comprises minor amounts of pollen, preferably less than 15 w % of pollen, even more preferably less than 10 w % of pollen, even more preferably less than 5 w % of pollen, even more preferably less than 1 w % of pollen;
optionally wherein the pollen supplement composition comprises one or more of:
   fatty acids comprising omega-6 and omega-3 fatty acids wherein the ratio of omega-6 fatty acids and omega-3 fatty acids is 5:1 to 1:20, preferably from 5:1 to 1:10, even more preferably from 2:1 to 1:5 (omega-6:omega-3) and wherein the omega-6 fatty acid preferably is linoleic acid and/or wherein the omega-3 fatty acid preferably is α-linolenic acid; and/or
   sterols in an amount from 0.01 w % to 4 w % wherein the sterols preferably are one or more of cholesterol, 24-methylene cholesterol, fucosterol, isofucosterol, campesterol, beta-sitosterol and stigmasterol; and even more preferably comprise all aforementioned sterols; and/or
   proanthocyanidins (multimers and/or monomers or their glucosides) in amount from 0.001 w % to 1 w %;
and wherein the w % is as compared to the total dry weight of the pollen supplement composition.

Another aspect of the present invention is a method for increasing bee performance, comprising:
providing a pollen supplement composition; and administering a pollen supplement composition to invertebrates of the Apidae families (bees), in particular honey bees (Apini), bumble bees (Bombini), or stingless honey bees (Meliponini);
wherein the pollen supplement composition is administered in the up to 3 months prior to the pollination period (this is the period when the forager bees are produced that will effectively dote pollination as well as the nurse bees that will feed the brood during the pollination)
and/or during the pollination period (additional nutrition is provided by nurse bees to the forager bees during this period by trophallaxis and new nurse bees and forager bees are produced) to bees deployed for pollination of the agricultural or horticultural crops susceptible of pollination by bees;
wherein the pollen supplement composition compensates for one or more dietary deficits in the pollen of the agricultural or horticultural crops; and
optionally wherein the pollen supplement composition comprises one or more of:
   fatty acids comprising omega-6 and omega-3 fatty acids wherein the ratio of omega-6 fatty acids and omega-3 fatty acids is 5:1 to 1:20, preferably from 5:1 to 1:10, even more preferably from 2:1 to 1:5 (omega-6:omega-3), and wherein the omega-6 fatty acid preferably is linoleic acid and/or wherein the omega-3 fatty acid preferably is α-linolenic acid;
   sterols in an amount from 0.01 w % to 4 w % wherein the sterols preferably are one or more of cholesterol, 24-methylene cholesterol, fucosterol, isofucosterol, campesterol, beta-sitosterol and stigmasterol; and even more preferably comprise all aforementioned sterols; and/or
   proanthocyanidins in amount from 0.001 w % to 1 w %;
wherein the pollen supplement composition is free of pollen or comprises minor amounts of pollen as an appetizer, preferably less than 15 w % of pollen, even more preferably less than 10 w % of pollen, even more preferably less than 5 w % of pollen, and
wherein the w % is as compared to the total dry weight of the pollen supplement composition providing agricultural or horticultural crops;

Another aspect of the present invention is a method for recovering performance of bees after the deployment for pollination of agricultural or horticultural crops susceptible of pollination by invertebrates of the Apidae families (bees), in particular honeybees (Apini), bumblebees (Bombini), or stingless honeybees (Meliponini), such as in a holding yard, comprising:
providing agricultural or horticultural crops;
providing one or more beehives within or in the immediate vicinity of the agricultural or horticultural crops to pollinate the agricultural or horticultural crops during the pollination period of the agricultural or horticultural crops or greenhouse crops or partially covered crops, in particular tunnel crops;
administering the pollen supplement composition during the pollination period (this is the period in which colony produces the forager and nurse bees that will be responsible in the recovery period to feed and nurse the brood and to forage for food) and after the pollination period to bees deployed for pollination of agricultural or horticultural crops susceptible of pollination by bees;
administering to the beehives a pollen supplement composition
   during a part or the full pollination period of the agricultural or horticultural crops; and/or
   during transportation from one crop to another crop; and/or
   in the holding yards;
wherein the pollen supplement composition compensates for one or more dietary deficits in the amount or quality of the pollen of the agricultural or horticultural crops; and/or in the environment;
wherein the pollen supplement composition is free of pollen or comprises minor amounts of pollen, preferably less than 15 w % of pollen, even more preferably less than 10 w % of pollen, even more preferably less than 5 w % of pollen, even more preferably less than 1 w % of pollen;
optionally wherein the pollen supplement composition comprises one or more of:
   fatty acids comprising omega-6 and omega-3 fatty acids wherein the ratio of omega-6 fatty acids and omega-3 fatty acids is 5:1 to 1:20, preferably from 5:1 to 1:10, even more preferably from 2:1 to 1:5 (omega-6:omega-3) and wherein the omega-6 fatty acid preferably is linoleic acid and/or wherein the omega-3 fatty acid preferably is α-linolenic acid; and/or
   sterols in an amount from 0.01 w % to 4 w % wherein the sterols preferably are one or more cholesterol, 24-methylene cholesterol, fucosterol, isofucosterol, campesterol, beta-sitosterol and stigmasterol; and even more preferably comprise all aforementioned sterols; and/or proanthocyanidins (multimers and/or monomers or their glucosides) in amount from 0.001 w % to 1 w %;
and wherein the w % is as compared to the total dry weight of the pollen supplement composition.

A further aspect of the present invention is a method for preparing bees for deployment in pollination of agricultural or horticultural crops susceptible of pollination by invertebrates of the Apidae families (bees), in particular honey bees (Apini), bumble bees (Bombini), or stingless honey bees (Meliponini), such as in a holding yard, comprising:
providing a pollen supplement composition; and
administering a pollen supplement composition to bees in advance of their deployment for pollination of agricultural or horticultural crops susceptible of pollination by bees or in advance of the recovery post pollination deployment;
providing agricultural or horticultural crops;
providing one or more beehives within or in the immediate vicinity of the agricultural or horticultural crops to pollinate the agricultural or horticultural crops during the pollination period of the agricultural or horticultural crops or greenhouse crops or partially covered crops, in particular tunnel crops;
administering to the beehives a pollen supplement composition
   during a part or the full pollination period of the agricultural or horticultural crops; and/or
   during transportation from one crop to another crop; and/or
   in the holding yards;
wherein the pollen supplement composition compensates for one or more dietary deficits in the amount or quality of the pollen of the agricultural or horticultural crops; and/or in the environment;
wherein the pollen supplement composition is free of pollen or comprises minor amounts of pollen, preferably less than 15 w % of pollen, even more preferably less than 10 w % of pollen, even more preferably less than 5 w % of pollen, even more preferably less than 1 w % of pollen;
optionally wherein the pollen supplement composition comprises one or more of: fatty acids comprising omega-6 and omega-3 fatty acids wherein the ratio of omega-6
   fatty acids and omega-3 fatty acids is 5:1 to 1:20, preferably from 5:1 to 1:10, even more preferably from 2:1 to 1:5 (omega-6:omega-3), and wherein the omega-6 fatty acid preferably is linoleic acid and/or wherein the omega-3 fatty acid preferably is α-linolenic acid; and/or
   sterols in an amount from 0.01 w % to 4 w % wherein the sterols preferably are one or more of cholesterol, 24-methylene cholesterol, fucosterol, isofucosterol, campesterol, beta-sitosterol and stigmasterol; and even more preferably comprise all aforementioned sterols; and/or
   proanthocyanidins (multimers and/or monomers or their glucosides) in amount from 0.001 w % to 1 w %;
and wherein the w % is as compared to the total dry weight of the pollen supplement composition.

In another aspect, the source of the proanthocyanidins is a non-pollen tissue of one or more of Solanaceae, Poaceae, Ranunculaceae, Fabaceae, Corylaceae, Cranberry/Blueberry; Apple (Rosaceae), Vit aceae.

In another aspect, the pollen supplement composition further comprises amino acids in an amount of 10 w % to 30 w %, preferably 15 w % to 25 w %, wherein the amount of essential amino acids is
10 w % to 15 w % arginine;
3 w % to 8 w % histidine;
1 w % to 5 w % isoleucine;
8 w % to 13 w % leucine;
40 w % to 50 w % lysine;
2 w % to 7 w % methionine;
3 w % to 8 w % phenylalanine;
4 w % to 9 w % threonine;
5 w % to 15 w % valine; and/or
5 w % to 10 w % tryptophan
as compared to the total of essential amino acids in the pollen supplement composition.

In another aspect, the pollen supplement composition further comprises one or more of
vitamins, preferably one or more of B1, B2, B3, B4, B5, B9, B7, B12, Inositol, A, C, E, Choline, preferably in a concentration of 0.0001 w % to 2 w %, preferably from 0.02 w % to 1 w %;
minerals, preferably one or more of zinc, iron, manganese, magnesium, copper, potassium, phosphorous, calcium and sodium;
wherein the w % is as compared to the total dry weight of the pollen supplement composition.

In another aspect, the background of the pollen supplement composition is as follows:
protein: 10 w % to 30 w %, preferably 15 w % to 25 w %,
carbohydrates, preferably sugars, even more preferably glucose, fructose or a mixture thereof: 0 w % to 70 w %, preferably 15 w % to 60 w %,
lipids comprising the sterols and fatty acids: 1 w % to 15 w %, preferably 5 w % to 10 w %; wherein the w % is as compared to the total dry weight of the pollen supplement composition.

In another aspect, the pollen supplement composition is as follows:
dry matter: 60 w % to 90 w %;
ash: 0.5 w % to 5 w %, preferably 1 w % to 4 w %; and
fiber: 0.1 w % to 5 w %, preferably 0.5 w % to 1 w %,
wherein the w % is as compared to the total dry weight of the pollen supplement composition.

In another aspect, the dry matter of the pollen supplement composition is 70 w % or more, preferably 75 w % or more, even more preferably 80 w % or more, wherein the w % is as compared to the total dry weight of the pollen supplement composition.

In another aspect the crop is a monoculture and wherein monoculture means a field cultivated with plants of one genus or one species of at least 0.2 ha, preferably of at least 1 ha, or 1-5 ha, preferably at least 5 ha, even more preferably at least 10 ha, even more preferably at least 50 ha, even more preferably at least 100 ha. In case the monoculture is gown in a greenhouse or a environment wherein the pollinators are contained by glass, plastic tunnel or area with insect netting, a field of any size (2 square meter to 10 ha). A field or greenhouse where some flowering plants are present or planted in between the rows of crop plants e.g. to feed the pollinators, is also defined as monoculture.

In another aspect, the agricultural crop is of one or more of the following families
Ericaceae, preferably blueberry (Vaccinium sect. Cyanococcus), or cranberry (Vaccinium subg. Oxycoccus)
Rosaceae, preferably almond (Prunus amygdalus), Apple (Malus domestica), cherry (Prunus avium), pear (Pyrus communis), raspberry (Rubus idaeus), blackberry (Rubus subg. Rubus)
Asteraceae, preferably sunflower (Helianthus annuus),
Brassicaceae, preferably canola (Brassica napus), Rapeseed (Brassica napus subsp napus),
Cabbage (red, green, broccoli, cauliflower, sprouts), (Brassica oleracea)
Lauraceae: Avocado
Cucurbitaceae, preferably cucumber (Cucumis sativus L), melon (Cucumis melo L), watermelon (Citrullus lanatus), calabash (Lagenaria siceraria), or pumpkin (Cucurbita spp.)
Umbellifers;
Apiaceae, preferably carrot (Daucus carota) or fennel (Foeniculum vulgare);
Amaryllidaceae, preferably onion (Allium cepa L)
Rutaceae, preferably citrus (Citrus L.),
Fabaceae, preferably alfalfa (Medicago sativa), soy (Glycine max), or peas (Lathyrus oleraceus).

In another aspect, the horticultural crop is of one or more of the following families:
Cucurbitaceae, preferably zucchini (Cucurbita pepo); or
Solanaceae, preferably aubergine (Solanum melongena).

In another aspect, the horticultural or agricultural crop is any crop wherein the purpose of planting the crop is to produce seed and whereby the crop is enclosed by a screen, glass or plastic construction (greenhouse, tent, tunnel) that prevents or limits foreign pollen or foreign pollinators to reach the crop.

In another aspect, the pollen supplement composition is free of or does only contain minor amounts, such as less than 5 w %, preferably less than 1 w %, even more preferably less than 0.1 w % of one or more of bee pheromones, sabinene, beta-pinene, limonene, nicotine, caffeine, floral fragrance components, isophorone and 4-oxoisophorone, citral, benzaldehyde, linalool, alpha-pinene, cinnamyl alcohol, and cis-3-hexenyl acetate.

In another aspect, the pollen supplement composition is administered in solid form such as a patty or in liquid form such as a solution or spray; inside or outside the beehive.

In another aspect, the increased bee performance and/or the increased pollination, crop quality and/or crop yield are obtained by one or more of:
reduced colony mortality;
increased number of bees produced;
increased number of capped brood;
increased bee population size;
improved bee fitness and vitality;
increased pollination performance;
increased vegetable quantity or fruit quality, preferably size, shape or form, increased vegetable quantity or fruit quantity, and
reduced rejection rate of vegetables or fruit, and increased seed quantity.

The inventors developed an effective method to protect bee colonies while deployed in the pollination of nutrition deficient crops and in conditions when the honeybees do not have access to sufficient and nutritionally adequate pollen, by feeding the colonies with a nutritionally adequate pollen-free artificial diet or a diet placed in the hive.

The inventors showed that a complete artificial diet is a viable option for the beekeeper to help colonies withstand and recover from these stressful events, such as before, during and after deployment for pollination. In a preferred embodiment, bee colonies are fed a pollen-free composition that is nutritionally balanced. This helps colonies sustain themselves during and recover more rapidly from such a deployment on nutritionally deficient crops or a nutritionally deficient environment.

### Deployment.

Deployment means any pollination event in commercial apiculture.

Deployment in particular means, serial deployment from February to September to pollinate different crops.

Deployment per crop typically is from 1-4 weeks per crop.

In one embodiment, deployment also includes transport between crops or between crops and holding yards.

In one embodiment, deployment also includes high-density storage in holding yards and honey production in deployment of crops.

In one embodiment, the methods of the present inventions address nutritional deficiencies of bees when pollinating a large area of a single crop that is not nutritionally suited for honeybees, hereinafter referred to as monoculture or monocrop. Examples include carrot, onion, squash, cucumber, melon, sunflower, blueberry.

In one embodiment, the methods of the present inventions address the lack pollen diversity in monocultures (32) (33).

In one embodiment, the methods of the present invention balance the effects of disease, pesticides (24) (25) (34), the stress from long distant transport (21), and the high density of storage in holding yard son colonies are well documented (24) (35) (36).

### Deployment in Blueberry and Sunflower Pollination.

In one embodiment, the methods of the present invention compensate for the reduction of colony populations during blueberry pollination, transport, and time spent in holding yard storage between pollination events in commercial beekeeping.

Blueberry pollen and nectar are usually not nutritious to honeybees).

The crude protein content of blue berry pollen is 13.9%, which is generally considered too low for sustained honeybee colony performance (Li et al. 2012 https://doi.org/10.1007/s13592-012-0126-0). This nutritional deficit could result in poor hive health (Dufour et al. 2020, https://doi.org/10.1371/journal.pone.02 27970), leaving bees more vulnerable to diseases such as European foul brood or sac brood virus (DOI 10.1093/jee/toae119), and making colonies less likely to be large enough for future pollination contracts. Understanding the for aging behavior of managed bees in blueberry farms can provide important information about the nutritional sources in these landscapes, and what floral resources may alleviate nutritional concerns.

Sunflower pollen has a low protein content and has a lower than the minimum required methionine and tryptophane content (two essential amino acids) and is deficient in arginine. (https://doi.org/10.1038/s41598-024-65569-1).

In one embodiment, the methods of the present invention, when deployed for blueberry pollination, compensate for one or more of the following issues:
the need for bee colonies to actively look for alternative forage,
reduced brood production,
decline in size,
subsequent deployment in sunflower, and/or
susceptibility to the bacterial diseases such as European foul-brood disease and/or viral diseases such as sac brood virus (37), DOI: 10.1093/jee/toae119).

Sunflower pollination has a similar, but less severe issue. Frequently colonies returning from serial pollination services are too weak to rebuild themselves on time for hibernation.

Accordingly, preferred deployments are in environments that are nutritionally deficient including one or more of blueberry and/or sunflower and/or almond orchards.

Other crops with nutrient deficiency or that are nutritious for honeybees are: cranberries, alfalfa (Medicago sativa), Mustard, Okra, Eucalyptus, Almonds, Rapeseed/canola, citrus trees, soybean, carrot, pears, kale.

### Serial Deployment.

In one embodiment, the deployment is a serial deployment.

Serial deployments are consecutive employments of beehives optionally including waiting times in holding periods.

Serial deployments leverage the different pollination periods of agricultural or horticultural crops. Examples include a serial deployment starting from blueberry followed by a holding period followed by a deployment to sunflower.

### Pollen Supplement Composition.

The feature “pollen supplement” means essentially free of pollen. However, minor amounts of pollen may be present in the compositions of the present inventions.

In one embodiment, the amount of pollen is 15 w % or less, preferably 10 w % or less, even more preferably 5 w % or less and even more preferably 1 w % or less and even more preferably 0.1 w % or less as compared to the dry weight of the pollen supplement composition.

The terms pollen substitute, pollen supplement and non-pollen are used interchangeably.

In another aspect, the pollen substitute composition comprises:
proteins in an amount from 10 w % to 50 w %, preferably of 15 w % to 40 w %,
fatty acids in an amount from 1 w % to 20 w %, preferably of 2 w % to 12 w %,
carbohydrates in an amount from 20 w % to 90 w %, preferably of 30 w % to 70 w %,
optionally vitamins, and
optionally minerals,
wherein the total amount of components adds up to 100 w % and wherein the w % are related to the total dry weight of the composition.

In a preferred embodiment, the pollen supplement composition comprises one or more of:
fatty acids comprising omega-6 and omega-3 fatty acids wherein the ratio of omega-6 fatty acids and omega-3 fatty acids is 5:1 to 1:20, preferably from 5:1 to 1:10, even more preferably from 2:1 to 1:5 (omega-6:omega-3) and wherein the omega-6 fatty acid preferably is linoleic acid and/or wherein the omega-3 fatty acid preferably is α-linolenic acid; and/or
sterols in an amount from 0.01 w % to 4 w % wherein the sterols preferably are one or more of cholesterol, 24-methylene cholesterol, fucosterol, isofucosterol, campesterol, beta-sitosterol and stigmasterol; and even more preferably comprise all aforementioned sterols; and/or
proanthocyanidins (multimers and/or monomers or their glucosides) in amount from 0.001 w % to 1 w %; and
wherein the w % is as compared to the total dry weight of the pollen supplement composition.

The ingredients of the pollen supplement composition may be obtained from pollen substitute sources for example through
synthetic chemical synthesis; or
any pollen substitute derived from an organism source
mineral sources.

Examples of pollen substitute organism sources of nutritionally relevant molecules (such as vitamins, amino acids, sterols, proteins, carbohydrates) include alga, fungus, bacteria, or animal parts that contain nutritionally relevant molecules.

In one embodiment, the pollen substitute sources naturally contain the nutritionally relevant ingredient, such as isofucosterol or 24-methylene cholesterol.

In another embodiment, the pollen supplement sources of isofucosterol are organisms metabolically engineered to produce the nutritionally relevant ingredient, such as isofucosterol or 24-methylene cholesterol.

### Dosage and Concentration.

In one embodiment, the dose and concentrations in the examples described below are for feeding a colony of 30000 bees with a 1.5 pound to 3 pounds of a bee patty over a period of 14 days.

The dose of the patty can be adapted to the size of the colony and the dose per 14 days can be repeated as desired by the beekeeper.

The same doses can be delivered in embodiments whereby the administration is not through patties but through other forms as described above. The formulae for calculating the dose for different formulations, methods of administration or size of bee colonies or as dose/day as % of insect biomass are described above.

In one embodiment, the isofucosterol or the fucosterol is administered in an amount that is nutritionally effective for invertebrates, in particular for honeybees or bumblebees.

In one embodiment, nutritionally effective involves feeding a colony of 30000 bees with 1 pound to 1.5 to 3 pounds of a bee patty formulation over a period of 14 days that has a concentration of isofucosterol or fucosterol or mixtures thereof in an amount from 0.01-5%, preferably from 0.01 to 2%, even more preferably from 0.01-1% as a percentage of the total weight of the pollen substitute composition.

In another embodiment, the concentration of isofucosterol or fucosterol or mixtures thereof is from 0.01-0.5% for honeybees or 0.01-1% for bumblebees as a percentage of the total weight of the pollen substitute composition.

The dose of the patty can be adapted to the size of the colony and the dose per 14 days can be repeated as desired by the beekeeper.

In one embodiment, the cholesterol is administered in an amount that is nutritionally effective for invertebrates, in particular for honeybees or bumblebees. Preferably the cholesterol is administered in a concentration of 0.001-2%, preferably 0.001%-1.5%, more preferably 0.01-1.2% by dry weight of the total pollen substitute composition.

In one embodiment, the 24-Methylenecholesterol is administered in an amount that is nutritionally effective for invertebrates, in particular for honeybees or bumblebees. Preferably the 24-Methylenecholesterol is administered in a concentration of 0.001-2%, preferably 0.001%-1.5%, more preferably 0.01-1.2% by dry weight of the total pollen substitute composition.

In one embodiment, the sitosterol is administered in an amount that is nutritionally effective for invertebrates, in particular for honeybees or bumblebees. Preferably the beta-sitosterol is administered in a concentration of 0.001-2%, preferably 0.001-1%, more preferably 0.03-0.6% by dry weight of the total pollen substitute composition.

In one embodiment, the campesterol is administered in an amount that is nutritionally effective for invertebrates, in particular for honeybees or bumblebees. Preferably the campesterol is administered in a concentration of 0.001-2%, preferably 0.001-1%, more preferably 0.02-0.35% by dry weight of the total pollen substitute composition.

In one embodiment, the stigmasterol is administered in an amount that is nutritionally effective for invertebrates, in particular for honeybees or bumblebees. Preferably the stigmasterol is administered in a concentration of 0.001-2%, preferably 0.001-1%, more preferably 0.01-2% by dry weight of the pollen substitute composition.

In one embodiment, the total concentration of sterol in the pollen substitute composition is in the range of 0.01% to 4% by dry weight of the pollen substitute composition.

### Dietary Deficits of Pollen.

In one embodiment, the dietary deficits of pollen mean an average protein content of less than 20 w % as compared to the total dry weight of the pollen.

### During the Pollination Period.

In one embodiment, the pollen supplement composition is administered during the deployment for pollination, for example for a period of 1 to 72 days, preferably 1 to 21 days, even more preferably 1 to 42 days.

### Before the Pollination Period.

In one embodiment, the pollen supplement composition is administered immediately before the deployment for pollination, for example for a period of 1 to 72 days, preferably 1 to 21 days, even more preferably 1 to 42 days in advance of the deployment for pollination. Typically, this concerns the holding yard and the transport to the field.

### After the Pollination Period.

In one embodiment, the pollen supplement composition is administered immediately after the deployment for pollination, for example for a period of 1 to 72 days, preferably 1 to 21 days, even more preferably 1 to 60 days after the deployment for pollination. Typically, this concerns the holding yard, the transport to the field and the deployment in nature or an agricultural environment where some pollen flow is present.

Crops and their Dietary Deficits.

There are several crops that are pollinated by bees, in particular honeybees that produce honey omectar that is nutritionally deficient for honeybees.

Typically, bees are deployed in conditions where the access to nutritionally adequate diverse pollen is limited. Examples include blueberries, cranberries, avocados, carrots, onions and sunflowers as well as many crops growing greenhouses such as melon and other cucurbits with nutritionally in adequate pollen.

In one embodiment, dietary deficits result from pollen that honeybees cannot easily harvest, pollen that has an outer wall (exine) with spines, pollen that is low in important nutrients (lipids, proteins, amino-acids, sterols), pollen of crop plants (sunflower) or plants in the environment that that contains toxins (sunflower, yellow jessamine).

In one embodiment, dietary deficits result from cucurbit pollen, such as zucchini, squash and cucumber pollen through its large, sticky and spiky nature. Moreover, cucurbit pollen may have compounds that are toxic to honeybees.

In one embodiment, dietary deficits result from sunflower pollen. Sunflower pollen is low in protein, spiky and contains delta-7 sterols and other compounds which are toxic to bees.

In one embodiment, dietary deficits result from blueberry pollen. Blueberry pollen has low acidity, and only 14 w % protein. This low protein content is considered too low for honeybees. Further, blueberry pollen is deficient in the essential amino-acids honeybees require. While deployed on blueberry crops, honeybees avoid collecting blueberry pollen and seek out pollen of other plant species.

In one embodiment, dietary deficits result from canola and/or sunflower. Canola and sunflower lack specific amino acids that are essential for bees, in particular for honeybees.

In one embodiment, dietary deficits result from avocado pollen. Avocado pollen is low in protein and lacks essential amino acids for bees, in particular for honeybees.

Dietary deficits resulting from deployment for pollination in monocultures.

In one embodiment, the dietary deficit results from a monoculture landscape where one or a few plant species are cultured.

A single or limited pollen source cannot fulfill the nutritional the nutritional needs of honeybees since honeybees require a diverse pollen source. Few, if any plants produce pollen that meets all nutritional needs of honeybees.

Bees, in particular honeybee colonies typically return from their deployment in monocrops for 4-6 weeks in a collapsing or collapsed state with a reduced number of frames of bees, low or no brood production and an increased bacterial (European foulbrood) and viral disease loads. This negative effect on the colony can perdure for months after such a deployment for pollination in monocultures.

In one embodiment, the methods of the present invention compensate for the dietary deficit and thus help the bee colonies recover and/or become large enough in the remainder of the summer season in order to be further deployed in other pollination or honey collection services.

In one embodiment, the methods of the present invention compensate for the dietary deficits associated with the deployment for pollination in monocrops and thus improve the size and vigor of the colony resulting in more colony splits and improved survival of bees and of the colony in the winter following such a deployment.

Consequently, the methods of the present invention present significant economic benefits for beekeepers.

For the growers it makes it easier to find beekeepers willing to place hives in their crops since the beekeepers benefit from a shorter recovery time of the colonies post-deployment and do not need to write them off after deployment.

Accordingly, in one embodiment, the methods of the present invention improve the degree and quality of pollination of the crops.

Bees.

The methods of the present invention apply to bees. Particularly preferred are bees of the Apidae or Bombidae family which are used as pollinators for agricultural or horticultural plants, such as
bees of the genus Apis and in particular Apis mellifera, Apis cerana, Apis dorsata, or
bumblebees of the genus Bombus and in particular Bombus terrestris, B. impatiens, B. ignites, or
stingless honeybees of the tribe Meliponini.

### Effect.

In one embodiment, the methods of the present invention help colonies withstand and recover from stressful events, such as before, during and after deployment for pollination as compared to the bees that were not administered a pollen supplement composition.

In one embodiment, the methods of the present invention improve key economic parameters relevant to commercial beekeepers, such as colony mortality, number of bees produced, and/or population size as compared to the bees that were not administered a pollen supplement composition.

In one embodiment, the methods of the present invention improve the rebound from stressful conditions, such as from deployment for pollination as compared to the bees that were not administered a pollen supplement composition.

In one embodiment, the methods of the present invention contribute to healthier, larger and more active bee colonies as compared to the bees that were not administered a pollen supplement composition.

In one embodiment, the methods of the present invention improve the pollination services of bees during deployment in agricultural or horticultural monocrops as compared to the bees that were not administered a pollen supplement composition.

In one embodiment, the methods of the present invention increase growers' yields and crop quality (32) (38) as compared to bees that were not administered a pollen supplement composition.

Increased Colony Size and/or Brood Production.

In one embodiment, the methods of the present invention led to improvements in bee longevity, flight capabilities, reduced behavior, and/or reduced homing and communication capabilities, herein after collectively referred to as “bee performance”.

### Increased Pollination, Crop Yield and Quality.

In one embodiment, the methods of the present invention led to improvements in the bee colonies' ability to withstand pathogens, pesticides, and abiotic stressors, and results in colonies that pollinate more efficiently resulting in higher crop yield and quality.

### SHORT DESCRIPTION OF THE DRAWINGS

FIG. 1: Overview of feeding experiments 1 and 2 of Example 1. Experiment 1 is a tent study and Experiment 2 a trial under field conditions. Colonies received the test diets from the start of the experiment (D 0-23 and 12 May 2023 for Experiments 1 and 2, respectively) to the last day (5 Oct. and 23 Sep. 2023, respectively). The figure also shows the assessment times relative to D0 (notepad symbols) and the estimated times of complete renewal of the capped brood (pupa symbols, every 12th day) and worker bee population (bee symbols, every 42nd day) based on average summer bee lifespans from literature (7) (8) (9).

FIG. 2A: Cumulative number of capped brood (FIG. 2A.1) and cumulative amount of feed consumed in hives (FIG. 2A.2) in tents fed diet D (a variant of diet A) compared a widely used commercial diet Diet E (Commercial Diet).

FIG. 2B: Cumulative number of capped brood (FIG. 2B.1) and cumulative amount of feed consumed (FIG. 2B.2) in hives in tents fed on Diet A compared to Diet D.

FIG. 2C: Capped brood count and average capped brood count in colonies fed Diet A (FIG. 2C.1) and Diet D (FIG. 2C.2) (starting Day 24) and capped brood and average capped brood count in colonies fed Diet D compared to Diet E (Commercial Diet) (2c.3) (starting Day 24). The brood produced between day 0-24 refers to brood produced with eggs laid prior to the start of the tent experiment and use of the reserves in the nurse bees to feed brood. Therefore, the brood produced between day 0 to 24 is not considered. Dotted lines represent individual colonies. Solid lines the average of the colonies.

FIG. 3: Diet A mitigates the serial stresses colonies experience in a typical commercial summer pollination cycle (Experiment 2). Over a 133-day period, 16 colonies per treatment group were serially placed in a holding yard (dense transit storage), commercial blueberry pollination fields and commercial sunflower seed production fields. P values indicate the statistical difference with the NF control at each time-point. Treatment groups were Diet A, Diet D, COMP (a widely used commercially available patty) and NF (no supplementary feed provided). X axis=time. (A) Average number of frames of bees per hive overtime. (B) Average amount of 5 cm2 areas of capped brood overtime. Vertical bars below the X-axis indicate feeding events.

FIG. 4: The 64 colonies were divided into 4 treatment groups (Diet A in orange, Diet D in blue, Diet COMP in grey and No Feed in yellow), with each group having 16 colonies. The hives were placed on 16 pallets (1 hive per treatment on each pallet) that were moved for pollination service from the holding yard to the blueberry field, then back to the yard, to the sunflower field and finally back to the yard.

FIG. 5: Colony-specific treatment effect on the count of frames of bees (Experiment 2). Bluelines correspond to the individual colonies, orange lines to the means.

FIG. 6: Changes in count of frames of bees (estimated marginal mean±95% CI) over the course of the trial (linear mixed effects model, Experiment 2)

FIG. 7: Colony-specific treatment effect on the number of 5 cm² squares of capped brood (Experiment 2). Blue lines correspond to the individual colonies, orange lines to the means.

FIG. 8: Changes in number of 5 cm² squams of capped brood (estimated marginal mean±95% CI) over the course of the trial (linear mixed effects model, Experiment 2).

### DETAILED DESCRIPTION

### Examples

### Example 1

In Experiment 1 of example 1, the inventors assessed the impact of two diets, (Diet A and Diet D) that are balanced for honeybees on nutritionally stressed honeybee colonies in the field by supplementary feeding colonies deployed in commercial pollination services during an entire summer. These colonies were deployed in blueberry pollination followed by sunflower seed pollination with intermittent storage in a holding yard, an environment with low pollen flow. Both blueberries (4); (5) and sunflower (6) are known to be nutritionally deficient for honeybees. Storage of colonies outside of the pollination events in dense bee holding yards with limited access to diverse pollen also stressful for honeybees.

The colonies received the Diet A, or Diet D (a diet with the same background as Diet A or no supplementary feed (NF), or Diet COMP which is a commercially available patty different from the commercially available patty used in Experiment 1.

Both Diet A and Diet D are considered nutritionally complete or nutritionally balanced for honeybees. Honeybee colonies were placed in tented enclosures and provided with Diet A or Diet D. These colonies stably produced brood for 120 days (>5 consecutive generations of brood production, 2.5 renewals of all honeybees in the colonies).

### Experimental Diets:

The inventors formulated a diet that includes the essential honeybee nutrients (reviews by (10) (3) (11)).

The inventors added the six sterols cholesterol, isofucosterol, 24-methylene cholesterol, beta-sitosterol, stigmasterol, campesterol in ratios and concentrations similar to those found in naturally foraging honeybees, at our research station in Belgium (Complete Diet A).

Samples of ingredients of the basal diet and sterol samples were analyzed for sterol composition using gas chromatography-mass spectroscopy (GC-MS, (1)) and the sterol concentrations of the diets calculated (Diet D has a similar background diet as Diet A but has a total sterol composition of 0.75% and a different sterol profile).

Two widely used commercial protein supplement diets were used as baseline: Diet E (Experiment 1; Reference available in EU and U.S.) and Diet COMP (Experiment 2; Available only in the U.S.). Experiment 2 also included a treatment wherein the bees received no supplementary protein patty (NF) and were fully dependent on foraging pollen.

Experiment 1: Tent study with no access to external food.

The inventors set up an assay with tent containing colonies with no access to external food. These colonies were fed pollen-free diets (treatments) with differing sterol compositions but an identical total sterol concentration, similar to that found in pollen and honey bees. The diets were formulated in the form of a patty and placed on top of the brood frames (standard beekeeping format).

Six colonies per treatment were placed indented enclosures and fed ad libitum one of three diets for 108 days.
Diet D the same diet as diet A but a different sterol composition and a higher total concentration of sterols.
Diet A: A pollen-free diet containing carbohydrates, proteins, lipids, minerals, vitamins and antioxidants and a trace of sterols, consisting of industrially available animal feed ingredients based on the US Patent application US20220361531A1
Diet E (Commercial Diet): Reference diet is a widely used commercial protein supplement.

Six small hives (type “Mini plus”, nucleus mating hives) per diet were populated with 800-850 ml (approximately 2000 bees) and a queen according to standard beekeeping practices.

The queens were freshly mated sister queens of identical age and genetic background (Apismellifera ligustica).

These colonies were kept outside to allow queen mating flights before the start of the study. These colonies were distributed over adjacent tent enclosures of 2 m×4.5 m×3 m, located in Belgium once it was confirmed that all queens started laying eggs and first larvae were present (Day 0=May 23rd).

Each tent contained three colonies, and two tents were foreseen per diet treatment group. As such, all three colonies within the same tent received the same diet throughout the course of the study.

On day 0 only traces of pollen and bee bread were present in the colonies. The diets were provided to each colony every 6 days by laying one or more patties of 50 grams on top of the brood frames and removing leftover diet as per standard beekeeping practice.

The dose was adapted over the course of the experiment to account for colony growth.

All colonies received an equal amount of their respective diet. The bees had ad libitum access to water and 50% sucrose sugar syrup in each enclosure. Water and sugar syrup were refreshed on a weekly basis.

Food consumption was measured every 6 days by weighing the leftover diets of each colony.

All brood frames were photographed every 12 days over 108 days, and the capped brood cells manually counted.

Since the capped brood stage lasts 12 days, all capped brood cells produced in a colony were counted once and only once. The capped brood observed on day 12 is derived from nutrition collected prior to D 0 and was not included in the analysis.

Any colonies with queens that stopped laying within the first 24 days were deemed to be queens that were not accepted at the creation of the colony and removed from the experiment.

The colonies experienced severe stressors during the experiment: starting the experiment with small freshly constituted colonies of young bees and a freshly mated queen, the confinement in tented enclosures, the disassembling of the colonies and the exposing of the brood to be photographed every 12 days to light and cold.

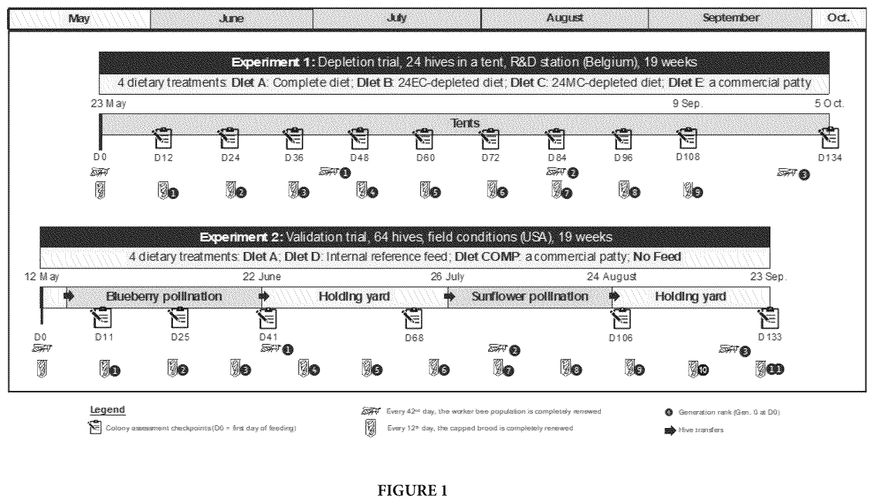

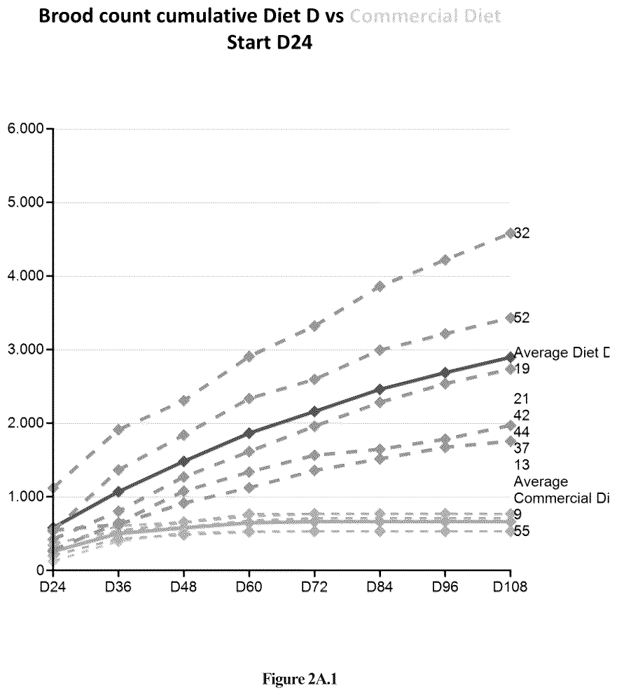

The time course of the experiment is shown in FIG. 1.

Six colonies per treatment were placed indented enclosures and fed ad libitum one of three diets for 108 days.

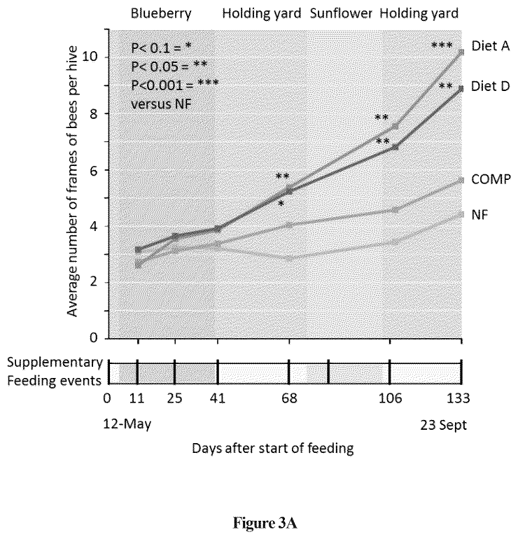

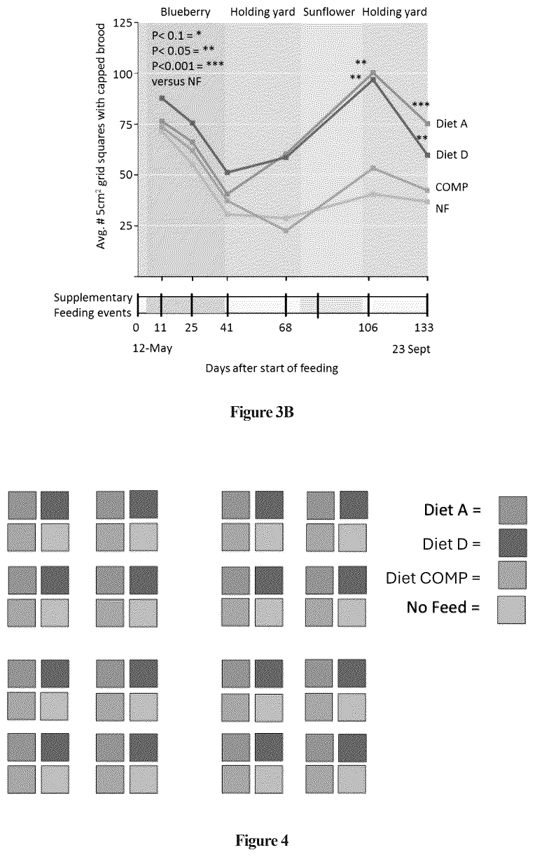

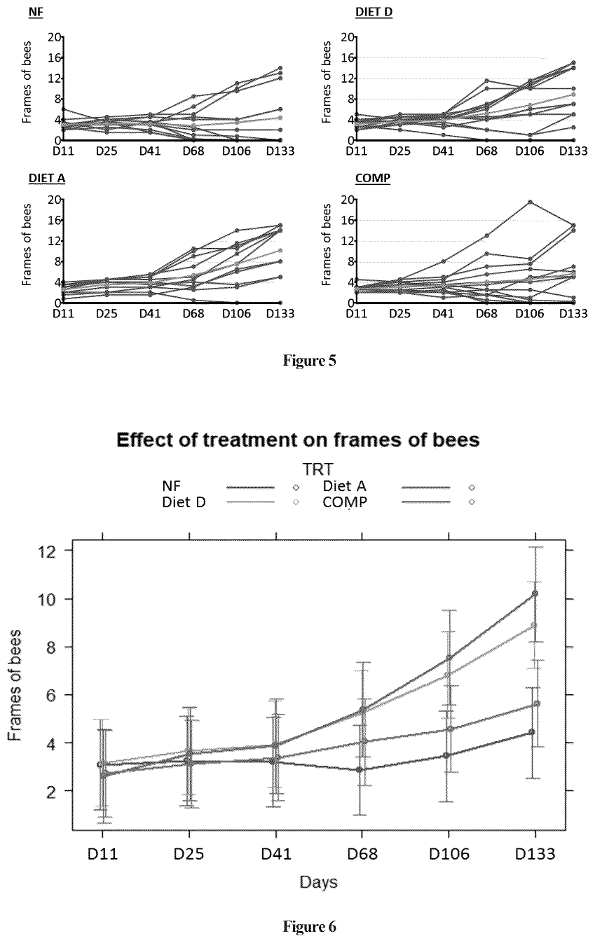

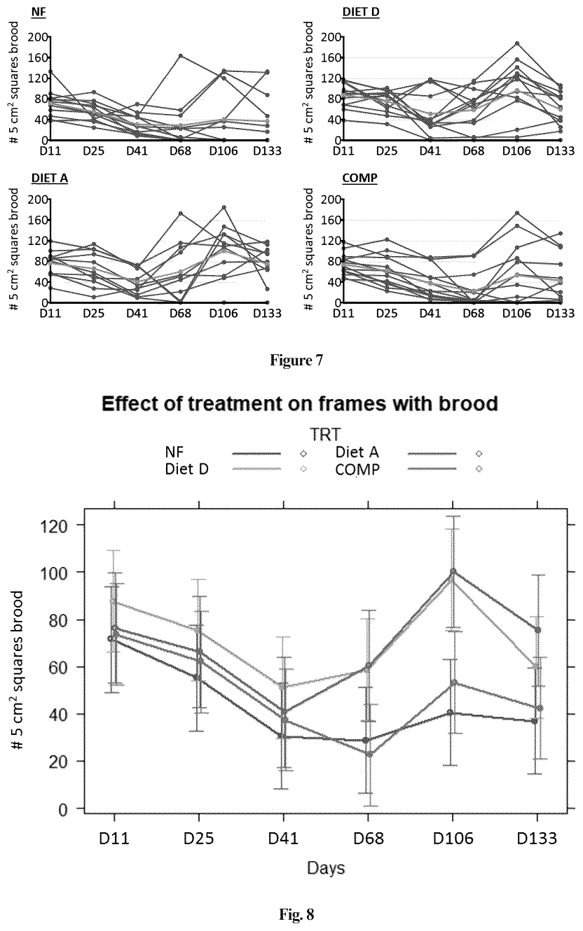

The bees had ad libitum access to water and 50% sucrose sugar syrup in each enclosure. Accounting for the 21-day brood cycle of honeybees, all capped brood present from day 24 onwards was produced from consumed artificial diets. Capped brood cells were counted every 12 days for all colonies and frames. The experimental design and results are presented in FIG. 3.

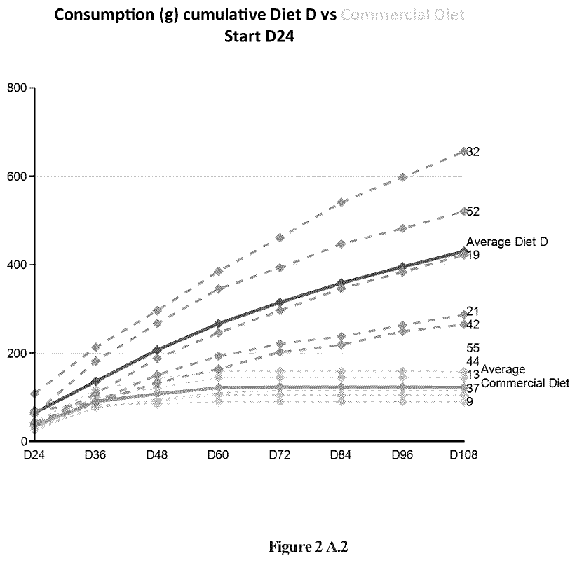

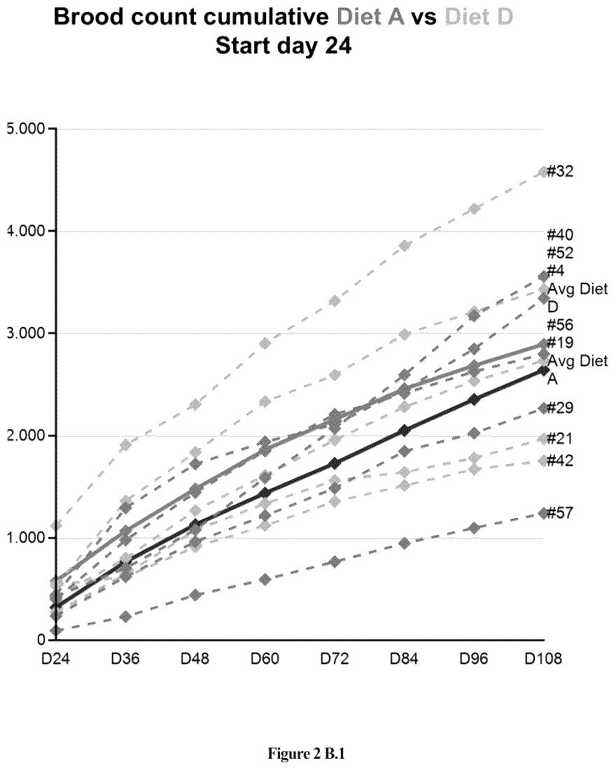

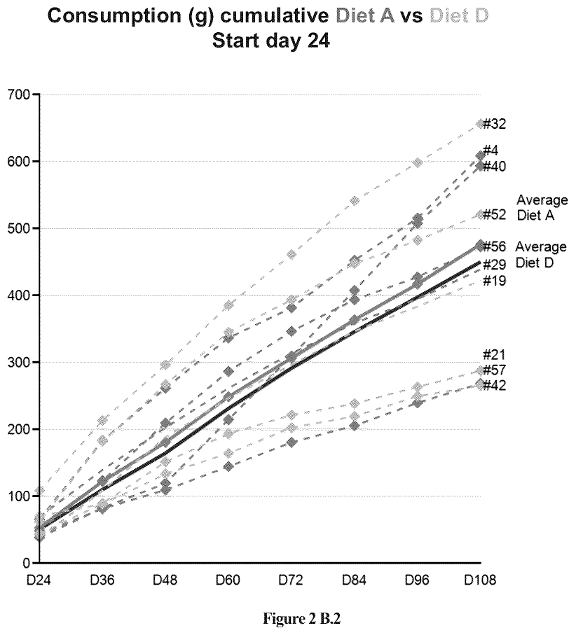

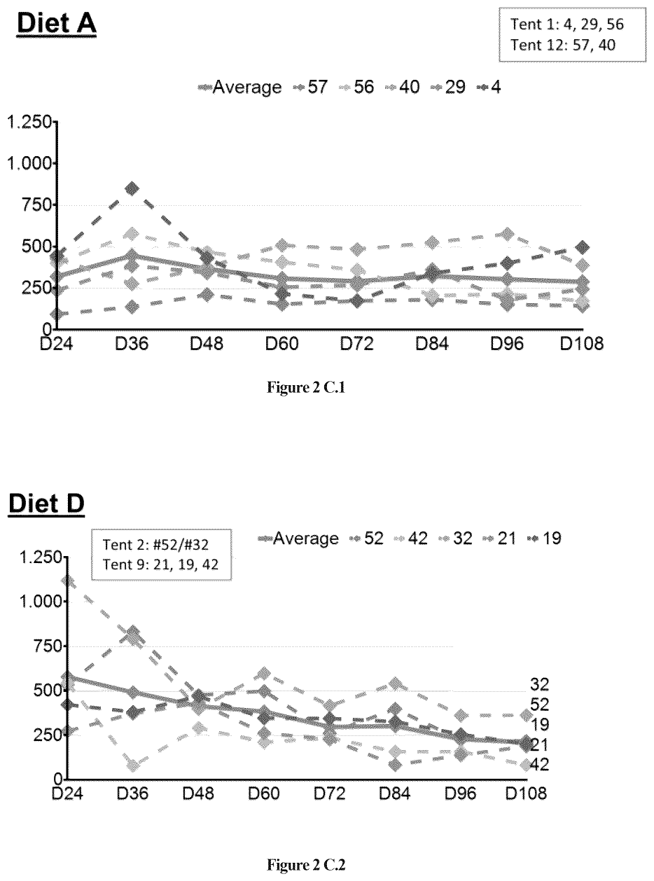

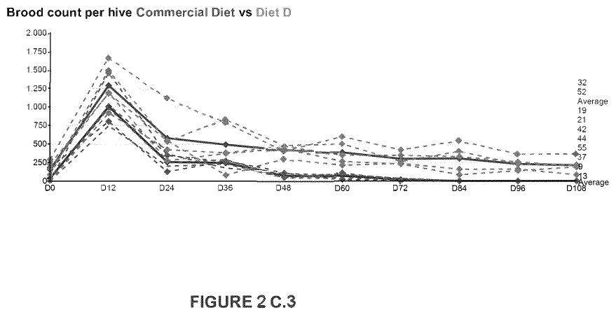

Colonies fed exclusively on Diet A and Diet D stably produced brood in the tent enclosures for at least 108 days (last date measured, FIG. 2). With an estimated average life span of a worker bee of ˜6 weeks (7) (8) (9), this 15-week duration represents two renewals of all worker bees in a colony, 9 consecutive brood cycles of 12 days and an estimated ˜5 renewals of the 24MC pool in honeybees (2).

Brood production in colonies fed a commercial protein supplement diet (Reference Diet E=Commercial Diet) collapsed rapidly. By day 48, these colonies produced on average 21% of the brood produced by Diet A and were essentially without brood on Day 72. This indicates that on this diet the bee colonies started lacking one or more specific nutrients after 36 days. The honeybees in colonies raised for 98/99 and 115 days on Diet E were uncoordinated and lethargic, while those raised on Diet A were normal.

The inventors furthermore conclude that unlike a leading commercial supplement reference, Diet A and Diet D are capable of sustaining honeybee colonies for at least 108 days—from May to September (5 consecutive generations of capped brood; 2.5 renewals of all bees in the colony), without any access to pollen, while colonies fed a leading protein supplement diet for honeybees collapse after 36 days.

To date no pollen-free artificial feed for honeybees has been produced that maintains brood production in colonies for more than 1 or 2 brood cycles (42 days).

Experiment set-up.

The inventors setup an assay with tent-contained colonies with no access to external food. These colonies were fed pollen-free diets (treatments). The diets were formulated in the form of a patty (standard beekeeping) and placed on top of the brood frames.

Six small hives (“Mini plus”, nucleus mating hives) per diet were populated with 800-850 ml bees (approximately 2,000 bees) and a queen according to standard beekeeping practices.

The queens were freshly mated sister queens of identical age and genetic background (Apismellifera ligustica).

These colonies were kept outside to allow queen mating flights before the start of the study. They were distributed over adjacent tent enclosures of 2 m×4.5 m×3 m with a concrete floor, located in Wingene, Belgium once it was confirmed that all queens started laying eggs (Day 0=May 23rd).

Each tent contained three colonies, and two tents were allocated pretreatment group. As such, all three colonies within the same tent received the same diet throughout the course of the study.

Varroa infection levels were monitored by examining the photographs of the frames for mites, and nosema by monitoring for brown diarrhea in combs and outside the hive. Varroa and nosema levels in the colonies were imperceptible in all treatments.

On Day 0 only traces of pollen and bee bread were present in the hives. The diets were provided to each colony every 6 days by laying one or more patties of 50 grams on top of the brood frames and removing leftover diet as per standard beekeeping practice.

Dose was adapted over the course of the experiment to account for colony growth. All colonies received an equal amount of their respective diet.

The bees had ad libitum access to water and 50% sucrose sugar syrup in each enclosure. Water and sugar syrup were refreshed on a weekly basis.

Feed consumption and brood production measurements:

Food consumption was measured every 6 days by weighing the leftover diets of each colony.

All brood frames were photographed every 12 days over 108 days, and the capped brood cells manually counted. Since the capped brood stage lasts 12 days, all capped brood cells produced in a colony were counted once and only once.

The capped brood observed on day 12 is derived from nutrition collected prior to D 0 and was not included in the trial data analysis. Accounting for the 21 day “egg to emerged honeybee” cycle of honeybees, all capped brood present from day 24 onwards was produced from consumed artificial diets. Any colonies with queens that stopped laying within the first 24 days were deemed to have queens that were not accepted at the creation of the colony and were removed from the experiment.

Egg laying of the queen tapers of as of mid-September (Day 108) in line with onset of the fall.

TABLE 1
Capped brood counts at each timepoint.

| Capped | | | | | | | | | | | | | % vs. | | % vs. |
|---|---|---|---|---|---|---|---|---|---|---|---|---|---|---|---|
| Brood | Hive | D | D | D | D | D | D | D | D | D | D | SumcappedbroodD | Diet | SumbroodcappedD | Diet |
| Count | # | 0 | 12 | 24 | 36 | 48 | 60 | 72 | 84 | 96 | 108 | 24-108 | A | 36-108 | A |
| Diet D | 19 | 0 | 917 | 421 | 380 | 467 | 345 | 343 | 325 | 254 | 198 | 2733 | | 2312 | |
| | 21 | 165 | 1186 | 273 | 371 | 431 | 261 | 226 | 82 | 137 | 187 | 1968 | | 1695 | |
| | 32 | 288 | 1661 | 1119 | 790 | 397 | 597 | 415 | 540 | 360 | 362 | 4580 | | 3461 | |
| | 42 | 227 | 1179 | 543 | 78 | 291 | 210 | 234 | 157 | 158 | 82 | 1753 | | 1210 | |
| | 52 | 84 | 1490 | 532 | 831 | 474 | 497 | 260 | 397 | 223 | 216 | 3430 | | 2898 | |
| Average | | 153 | 1287 | 578 | 490 | 412 | 382 | 296 | 300 | 226 | 209 | 2893 | 110% | 2315 | 100% |
| Commercial | 9 | 0 | 743 | 199 | 229 | 52 | 41 | 9 | 0 | 0 | 0 | 530 | | 331 | |
| Diet | 13 | 0 | 982 | 245 | 287 | 54 | 88 | 33 | 1 | 0 | 0 | 708 | | 463 | |
| | 37 | 0 | 1020 | 364 | 174 | 108 | 86 | 34 | 0 | 0 | 0 | 766 | | 402 | |
| | 44 | 0 | 1460 | 344 | 258 | 54 | 109 | 4 | 0 | 0 | 0 | 769 | | 425 | |
| | 55 | 181 | 801 | 124 | 270 | 116 | 19 | 0 | 0 | 0 | 0 | 529 | | 405 | |
| Average | | 36 | 1001 | 255 | 244 | 77 | 69 | 16 | 0 | 0 | 0 | 660 | 25% | 405 | 17% |
| Diet A | 4 | 0 | 1210 | 443 | 849 | 430 | 215 | 172 | 339 | 399 | 495 | 3342 | | 2899 | |
| | 29 | 0 | 1022 | 236 | 384 | 342 | 255 | 268 | 361 | 178 | 243 | 2267 | | 2031 | |
| | 40 | 290 | 1003 | 427 | 276 | 375 | 508 | 483 | 525 | 576 | 387 | 3557 | | 3130 | |
| | 56 | 0 | 1612 | 400 | 578 | 465 | 406 | 359 | 204 | 213 | 172 | 2797 | | 2397 | |
| | 57 | 0 | 1095 | 93 | 137 | 210 | 153 | 173 | 180 | 151 | 143 | 1240 | | 1147 | |
| Average | | 58 | 1188 | 320 | 445 | 364 | 307 | 291 | 322 | 303 | 288 | 2641 | 100% | 2321 | 100% |

TABLE 2
Feed consumed and feed conversion ratio at each timepoint.

| | | | | | | | | | | | | | | | | | | | | | Milligram |
|---|---|---|---|---|---|---|---|---|---|---|---|---|---|---|---|---|---|---|---|---|---|
| Consumption | | | | | | | | | | | | | | | | | | | | Sum | diet |
| Data: g | Hive | D | D | D | D | D | D | D | D | D | D | D | D | D | D | D | D | D | D | consumption | eaten/brood |
| per 6 days | nr | 6 | 12 | 18 | 24 | 30 | 36 | 42 | 48 | 54 | 60 | 66 | 72 | 78 | 84 | 90 | 96 | 102 | 108 | Day 12-108 | made |
| Diet D | 19 | 14 | 36 | 28 | 38 | 34 | 36 | 35 | 45 | 28 | 30 | 22 | 28 | 21 | 29 | 18 | 19 | 20 | 19 | 486 | 178 |
| | 21 | 0 | 1 | 29 | 43 | 27 | 19 | 30 | 32 | 21 | 21 | 19 | 9 | 4 | 13 | 7 | 18 | 12 | 12 | 317 | 161 |
| | 32 | 25 | 31 | 95 | 108 | 61 | 44 | 35 | 48 | 54 | 35 | 33 | 43 | 38 | 42 | 26 | 31 | 29 | 29 | 782 | 171 |
| | 42 | 1 | 1 | 45 | 70 | 9 | 9 | 21 | 24 | 13 | 18 | 17 | 21 | 9 | 8 | 16 | 14 | 7 | 9 | 311 | 177 |
| | 52 | 1 | 1 | 25 | 62 | 66 | 54 | 44 | 41 | 49 | 29 | 17 | 31 | 25 | 29 | 16 | 19 | 18 | 21 | 547 | 159 |
| Average | | 8 | 14 | 44 | 64 | 39 | 32 | 33 | 38 | 33 | 27 | 22 | 26 | 19 | 24 | 17 | 20 | 17 | 18 | 489 | 169 |
| CommercialDiet | 9 | 21 | 46 | 8 | 33 | 29 | 21 | 0 | 2 | 4 | 1 | 0 | 0 | 0 | 0 | 0 | 0 | 0 | 0 | 144 | 272 |
| | 13 | 58 | 54 | 21 | 30 | 26 | 19 | 9 | 12 | 10 | 4 | 4 | 1 | 0 | 0 | 0 | 0 | 0 | 0 | 190 | 268 |
| | 37 | 32 | 41 | 21 | 25 | 24 | 28 | 3 | 12 | 7 | 6 | 0 | 0 | 0 | 0 | 0 | 0 | 0 | 0 | 167 | 218 |
| | 44 | 46 | 32 | 21 | 40 | 38 | 22 | 8 | 13 | 20 | 4 | 0 | 0 | 0 | 0 | 0 | 0 | 0 | 0 | 198 | 257 |
| | 55 | 33 | 10 | 27 | 44 | 35 | 38 | 17 | 10 | 12 | 3 | 0 | 0 | 0 | 0 | 0 | 0 | 0 | 0 | 196 | 371 |
| Average | | 38 | 37 | 20 | 34 | 30 | 26 | 7 | 10 | 11 | 4 | 1 | 0 | 0 | 0 | 0 | 0 | 0 | 0 | 179 | 271 |
| Diet A | 4 | 1 | 13 | 31 | 65 | 56 | 62 | 41 | 37 | 45 | 30 | 22 | 23 | 27 | 44 | 38 | 25 | 43 | 50 | 652 | 195 |
| | 29 | 0 | 0 | 11 | 69 | 34 | 36 | 35 | 28 | 34 | 25 | 19 | 33 | 29 | 16 | 18 | 18 | 22 | 22 | 449 | 198 |
| | 40 | 17 | 26 | 65 | 38 | 28 | 16 | 31 | 6 | 50 | 45 | 46 | 49 | 47 | 51 | 56 | 44 | 42 | 44 | 684 | 192 |
| | 56 | 4 | 5 | 36 | 48 | 35 | 40 | 44 | 42 | 42 | 35 | 28 | 32 | 22 | 25 | 20 | 14 | 14 | 31 | 513 | 183 |
| | 57 | 1 | 1 | 18 | 43 | 26 | 12 | 13 | 15 | 15 | 20 | 16 | 20 | 12 | 13 | 17 | 17 | 16 | 13 | 287 | 231 |
| Average | | 5 | 9 | 32 | 53 | 36 | 33 | 33 | 26 | 37 | 31 | 26 | 31 | 27 | 30 | 30 | 24 | 27 | 32 | 517 | 196 |

Experiment 2: Field study with serial stressors.

In the Spring of 2023, 64 colonies were established in Othello Washington (USA), by installing 1.36 kg (3 lbs.) bee packages into one 8-frame hive body box with provided honey frame and kept on standard 8-frame hive pallets.

The sister queens from each package were caged and were allowed to be released by the worker bees.

The newly established colonies were divided into four groups: Diet A, Diet D, COMP, and Faith each group having 16 colonies at start.

On May 12 (Day 0), the colonies had a pre-assessment feeding and on Day 5 all hives were moved from the holding yard to commercial blueberries fields in Western Washington State. On Day 41 they were moved back to the holding yard and from there on D75 to commercial sunflower seed production fields. On Day 104 they were returned to the holding yard.

Hive management followed standard Washington State University beekeeping practices and additional hive boxes were added as needed on an individual hive level.

Assessments were performed on days 11, 25, 41, 68, 106 and 133. The number of frames of bees was recorded visually and the area of capped brood was measured using a 5 cm² grid.

The queen status was also recorded, mite levels were measured, and adult bee samples were collected for further molecular analysis (data not shown).

Colonies were fed on days 0, 11, 25, 41, 68, 83, and 95. The diets were formulated in the form of a patty and placed on top of the brood frames (standard beekeeping practice). Any remaining feed was collected, and new feed was given to the colonies for their corresponding diets.

All patties weighed roughly 12 oz, and COMP patties were reduced to match this size.

In feeding events prior to July 1st, all colonies were given one patty. In feeding events after July 1st, colonies that had no remaining patty from the previous feeding were given two patties, and all other colonies were given one patty.

The inventors excluded any colonies with signs of queen failure or non-acceptance and diseases prior to day 41, which the inventors put down to failures in hive set-up and queen acceptance.

The blueberry fields were in North-Western Washington State, near the town of Mount Vernon, WA. This is an area described as warm-summer Mediterranean under the Köppen climate classification.

The sunflower pollination fields and the holding yard were both located in Eastern Washington State, near the town of Othello. These fields were roughly 8 km one from another and are in an area described as cold semi-arid under the Köppen climate classification.

### Statistical Methods for Differences in Frames of Bees and Frames of Capped Brood.

The main objective of the statistical analysis was to compare treatment effects on counts and cumulative counts of capped brood cells over the course of the experiment.

Colony-specific changes in capped brood cells were compared between treatments. Treatment and time and their interaction were used as fixed factors and colonies as random effects in a linear mixed effects model (LMEM).

All analyses were performed in the R version 4.3.1 (12) using the lme4 package (13). Systematic departures from the normality assumptions were verified using QQ-plots, scatterplots of the residuals versus the predictions were used to check for systematic departures from the mean model. A value of p<0.05 was considered statistically significant.

The colony-specific effect of the treatment on the cumulative counts of capped brood cells was investigated using a linear mixed effects model. A linear mixed effects model was fitted to the data.

Treatment, time and their interaction were used as fixed factors and hives as random effects.

Additional information on the statistical methods is available in the Supplemental Material.

### Results

Supplementary feeding with an artificial pollen-replacing diet mitigates stress during serial blueberry and sunflower pollination with intermittent holding yard storage and avoids colony collapse (Experiment 2).

Over the 4.3-month period of Experiment 2, the colonies that were fed Diets A and D grew from ˜3 frames of bees to an average of 10.2 and 8.9 frames of bees, respectively (FIG. 3A).

The COMP and NF colonies reached about half this size (average of 5.6 and 4.4 frames of bees respectively).

A linear mixed effects model (LMEM) was fitted to the data to assess differences in frames of bees of the diet treatments over time (see Methods).

Until Day 41, no significant differences in changes from the start were observed between the diets (D, A, COMP) and the NF group.

For Diet A, a significant treatment effect versus the NF group was observed from Day 68 onwards: the estimated number of frames of bees was on average 3.0 frames of bees higher than the NF colonies on Day 68 (95% CI: 0.29, 5.70, p=0.030). This difference increased to 4.6 frames of bees on Day 106 (95% CI: 1.87, 7.29, p=0.001) and 6.2 frames of bees on Day 133 (95% CI: 3.53, 8.94, p<0.001).

From Day 106 onwards Diet D colonies also showed a statistically significant increase in the estimated number of frames of bees compared to the NF group: on average 3.3 frames of bees higher for Day 106 (95% CI: 0.68, 5.88, p=0.014) and 4.4 higher for Day 133 (95% CI: 1.78, 6.98, p=0.001). In contrast, COMP colonies did not statistically differ in frames of bees from the NF group across time (p>0.05).

On Day 133, 73% (8/11 hives) of the Diet A colonies and 69% (9/13 hives) of the Diet D colonies were larger than 6 frames of bees compared to 31% (4/13 hives) in the COMP colonies and 24% (3/12 hives) in NF group. Colonies smaller than 6 frames of bees are unlikely to successfully overwinter and are considered too small to serve in pollination services.

Diet A and D colonies produced a higher amount of capped brood compared to COMP feed and NF group (FIG. 3B). A LMEM was fitted to the capped brood count data (see Methods). In the period that the hives were in blueberry fields, the amount of capped brood was statistically identical across the four treatments and decreasing, consistent with beekeeper's experiences that colonies decline in blueberry fields.

However, as soon as the hives were out of blueberry fields, Diet A and Diet D colonies immediately and steadily increased brood production from Day 68 until Day 106 (the end of August). They showed a reduced capped brood area at the final assessment on Day 133 (22nd of September), consistent with the natural annual colony cycle whereby queens reduce egg-laying in the fall. COMP colonies did not statistically differ in capped brood area from the NF group across time (p>0.05).

NF and COMP hives continued to show reducing capped brood numbers until Day 68 and only showed a recovery in capped brood ama on Day 106. COMP and NF had on Days 68, 106 and 133 about half the brood present compared to Diets A and D.

On Day 68, the Diet A and Diet D colonies had an average of 60 and 59 5 cm² areas of capped brood, versus 22 for COMP and 29 for NF colonies. On Day 106 this was 100 for the Diet A colonies and 97 for the Diet D colonies, versus 53 for COMP and 40 for NF colonies. On Day 133 this was 75 for Diet A and 60 for Diet D, versus 42 for COMP and 37 for NF.

Diet A and Diet D colonies had a mortality of respectively 9% (1/11 hives) and 8% (1/13 hives) compared to 23% (3/13 hives) for COMP colonies and 50% (6/12 hives) for NF colonies by Day 133 (see Supplemental material).

The inventors placed colonies in stressful commercial field conditions and tested whether colonies fed Diet A were more able to resist malnourishment and other stress such as transport and pollination in commercial fields on crops that are nutritionally deficient for honeybees—blueberries and sunflowers. Colonies receiving no feed or the commercially purchased pollen supplement were not statistically different from one another and did not grow enough in these conditions to survive the winter.

However, Diet A and Diet D enabled honeybee colonies to grow from May to September, through periods of commercial pollination on nutrient deficient crops and storage in bee-yards, when the natural pollen flow is inadequate, and conditions are stressful. The inventors conclude that sustained supplementary feeding of Diet A and Diet D prevented colony collapse in field conditions while NF and a currently leading commercial protein supplement did not.

### Example 3

In 9 different experiments Diet D was fed to colonies deployed in glass greenhouses in Belgium to pollinate crops were grown for produce production (Zucchini) or seed production (Cruciferae).

An equal number of hives fed with Apix patties=Diet D and hives that did not receive supplementary feeding was placed in each greenhouse, see Table 4 for a summary of the experiments.

In these greenhouse settings the honeybees are highly stressed since they only have a single source of pollen in inadequate quantity for the size of the hives placed in the greenhouse. They furthermore are chronically exposed to fungicides and insecticides and hive a high death rate due to collision with the greenhouse glass surfaces. Zucchini (Cucurbit) pollen is known to be nutritionally deficient for honeybees, sticky and spiny.

TABLE 4
Summary of the Experimental design of Experiment 1 to 9 of Example 3.

| Experiment | | | | | | | |
|---|---|---|---|---|---|---|---|
| nr | Purpose | Situation | Crop | Location(Belgium) | #Hivesfed | #Controlhives | Duration |
| 1 | Pollination, | Greenhouse | Zucchini | Antwerp | 3 | 3 | 3 weeks |
| | fruitsetting | | | | | | |
| 2 | Pollination, | Greenhouse | Zucchini | Antwerp | 3 | 3 | 12 weeks |
| | fruitsetting | | | | | | |
| 3 | Pollination, | Greenhouse | Zucchini | Roeselare | 2 | 2 | 12 weeks |
| | fruitsetting | | | | | | |
| 4 | Pollination, | Greenhouse | Zucchini | Roeselare | 2 | 2 | 12 weeks |
| | fruitsetting | | | | | | |
| 5 | Pollination, | Greenhouse | Zucchini | Staden | 3 | 3 | 12 weeks |
| | fruitsetting | | | | | | |
| 6 | Pollination, | Greenhouse | Cauliflower | Drongen | 5 | 5 | 3 weeks |
| | seedproduction | | | | | | |
| 7 | Pollination, | Greenhouse | Broccoli | Dendermonde | 4 | 4 | 3 weeks |
| | seedproduction | | | | | | |
| 8 | Pollination, | Greenhouse | White cabbage | Hamme | 9 | 9 | 4 weeks |
| | seedproduction | | | | | | |
| 9 | Pollination, | Greenhouse | Red cabbage | Dendermonde | 2 | 2 | 3 weeks |
| | seedproduction | | | | | | |

TABLE 5
Summaryoftheexperimentalprotocol(feedingdoseandtiming,
timewindowthehivesweredeployedinthegreenhousesandtime-
pointwhenthehiveswereplacedinadiscoveryyard(APIXpatties = Diet D).

| | | Jan | Feb | | Mar | | | Apr | | | | May | | | | Jun | |
|---|---|---|---|---|---|---|---|---|---|---|---|---|---|---|---|---|---|
| Experiment | Feed Apix patties | X | | X | | X | | | X | | | | | | | | |
| 1: | 3 × 340 g/hive | | | | | | | | | | | | | | | | |
| | Greenhouse | | | | | X | X | X | | | | | | | | | |
| | Recovery yard | | | | | | | | X | | | | | | | | |
| Experiment | Feed Apix patties | X | X | | X | X | | X | | | | | | | | | |
| 2: | 3 × 340 g/hive | | | | | | | | | | | | | | | | |
| | Greenhouse | | | | | | | X | X | X | X | X | X | X | X | X | X |
| | Recovery yard | | | | | | | | | | | | | | | | |
| Experiment | Feed Apix patties | X | X | | X | | | X | | | | | | | | | |
| 3: | 3 × 340 g/hive | | | | | | | | | | | | | | | | |
| | Greenhouse | | | | | | | X | X | X | X | X | X | X | X | X | X |
| | Recovery yard | | | | | | | | | | | | | | | | |
| Experiment | Feed Apix patties | X | X | | X | | | X | | | | | | | | | |
| 4: | 3 × 340 g/hive | | | | | | | | | | | | | | | | |
| | Greenhouse | | | | | | | X | | | | | | | | | |
| | Recovery yard | | | | | | | | | | | | | | | | |
| Experiment | Feed Apix patties | X | X | | X | | | X | | | | | | | | | |
| 5: | 3 × 340 g/hive | | | | | | | | | | | | | | | | |
| | Greenhouse | | | | | | | X | X | X | X | X | X | X | X | X | X |
| | Recovery yard | | | | | | | | | | | | | | | | |
| Experiment | Feed Apix patties | X | X | | X | | X | | | X | | | | | | | |
| 6: | 3 × 340 g/hive | | | | | | | | | | | | | | | | |
| | Greenhouse | | | | | | | | | X | X | X | | | | | |
| | Recovery yard | | | | | | | | | | | | X | | | | |
| Experiment | Feed Apix patties | X | X | | X | | X | X | | | X | | | | | | |
| 7: | 3 × 340 g/hive | | | | | | | | | | | | | | | | |
| | Greenhouse | | | | | | | | | | X | X | X | | | | |
| | Recovery yard | | | | | | | | | | | | | X | | | |
| Experiment | Feed Apix patties | X | X | | X | | X | | X | | | | | | | | |
| 8: | 3 × 340 g/hive | | | | | | | | | | | | | | | | |
| | Greenhouse | | | | | | | | X | X | X | X | | | | | |
| | Recovery yard | | | | | | | | | | | | | | | | |
| Experiment | Feed Apix patties | X | X | | X | | X | | X | | | | | | | | |
| 9: | 3 × 340 g/hive | | | | | | | | | | | | | | | | |
| | Greenhouse | | | | | | | | X | X | X | | | | | | |
| | Recovery yard | | | | | | | | | | | X | | | | | |

| | | Jun | | Jul | | | | Aug | | | | Sept | | | | Oct |
|---|---|---|---|---|---|---|---|---|---|---|---|---|---|---|---|---|
| Experiment | Feed Apix patties | | | | | | | | | | | | | | | |
| 1: | 3 × 340 g/hive | | | | | | | | | | | | | | | |
| | Greenhouse | | | | | | | | | | | | | | | |
| | Recovery yard | | | | | | | | | | | | | | | |
| Experiment | Feed Apix patties | | | X | | | | | | | | | | | | |
| 2: | 3 × 340 g/hive | | | | | | | | | | | | | | | |
| | Greenhouse | X | X | | | | | | | | | | | | | |
| | Recovery yard | | | X | | | | | | | | | | | | |
| Experiment | Feed Apix patties | | | X | | | | | | | | | | | | |
| 3: | 3 × 340 g/hive | | | | | | | | | | | | | | | |
| | Greenhouse | X | X | | | | | | | | | | | | | |
| | Recovery yard | | | X | | | | | | | | | | | | |
| Experiment | Feed Apix patties | | | X | | | | | | | | | | | | X |
| 4: | 3 × 340 g/hive | | | | | | | | | | | | | | | |
| | Greenhouse | | | X | X | X | X | X | X | X | X | X | X | X | X | |
| | Recovery yard | | | | | | | | | | | | | | | X |
| Experiment | Feed Apix patties | | | X | | | | | | | | | | | | |
| 5: | 3 × 340 g/hive | | | | | | | | | | | | | | | |
| | Greenhouse | X | X | | | | | | | | | | | | | |
| | Recovery yard | | | X | | | | | | | | | | | | |
| Experiment | Feed Apix patties | | | | | | | | | | | | | | | |
| 6: | 3 × 340 g/hive | | | | | | | | | | | | | | | |
| | Greenhouse | | | | | | | | | | | | | | | |
| | Recovery yard | | | | | | | | | | | | | | | |
| Experiment | Feed Apix patties | | | | | | | | | | | | | | | |
| 7: | 3 × 340 g/hive | | | | | | | | | | | | | | | |
| | Greenhouse | | | | | | | | | | | | | | | |
| | Recovery yard | | | | | | | | | | | | | | | |
| Experiment | Feed Apix patties | | | | | | | | | | | | | | | |
| 8: | 3 × 340 g/hive | | | | | | | | | | | | | | | |
| | Greenhouse | | | | | | | | | | | | | | | |
| | Recovery yard | | | | | | | | | | | | | | | |
| Experiment | Feed Apix patties | | | | | | | | | | | | | | | |
| 9: | 3 × 340 g/hive | | | | | | | | | | | | | | | |
| | Greenhouse | | | | | | | | | | | | | | | |
| | Recovery yard | | | | | | | | | | | | | | | |

TABLE 6
Summary of the result of Experiments 1-9 of Example 3.

| | | | Total | Total | #frames | #frames | #frames | #frames | Presence of | Bees | Activity |
|---|---|---|---|---|---|---|---|---|---|---|---|
| Experiment | Apix | Hive | nr of | nr of | of bees | of bees | of brood | of brood | paralytic | bearding | at leaving |
| nr | patties | type | supers | frames | @entering | @leaving | @entering | @leaving | virus | outside hive | greenhouse |
| 1 | + | simplex | 2 | 22 | 22 | 10 to 11 | 8 | 2 | + | + | − |
| | − | simplex | 2 | 22 | 22 | 5 | 8 | 1 | + | − | − |
| 2 | + | simplex | 2 | 22 | 22 | 22 | 15 | 6 | + | + | +/− |
| | − | simplex | 2 | 22 | 22 | 10 to 11 | 15 | 3 | + | − | − |
| 3 | + | simplex | 2 | 22 | 22 | 22 | 15 | 6 | + | + | +/− |
| | − | simplex | 2 | 22 | 22 | 10 to 11 | 15 | 3 | + | − | − |
| 4 | + | simplex | 2 | 22 | 22 | 22 | 15 | 6 | + | + | +/− |
| | − | simplex | 2 | 22 | 22 | 10 to 11 | 15 | 3 | + | − | − |
| 5 | + | simplex | 2 | 22 | 22 | 22 | 15 | 6 | + | + | +/− |
| | − | simplex | 2 | 22 | 22 | 10 to 11 | 15 | 3 | + | − | − |
| 6 | + | simplex | 2 | 22 | 22 | 22 | 12 | 12 | + | + | +/− |
| | − | simplex | 2 | 22 | 22 | 15 | 12 | 6 to 7 | + | − | − |
| 7 | + | simplex | 2 | 22 | 22 | 22 | 12 | 12 | + | + | +/− |
| | − | simplex | 2 | 22 | 22 | 15 | 12 | 6 to 7 | + | − | − |
| 8 | + | simplex | 2 | 22 | 22 | 22 | 12 | 12 | + | + | +/− |
| | − | simplex | 2 | 22 | 22 | 15 | 12 | 6to 7 | + | − | − |
| 9 | + | simplex | 2 | 22 | 22 | 22 | 12 | 12 | + | + | +/− |
| | − | simplex | 2 | 22 | 22 | 15 | 12 | 6 to 7 | + | − | − |

+ = fed artificial Apix patties (Diet D = Apix patties); − = no patties fed.

The number of frames of bees and the number of frames of brood for each hive was measured at the start of the experiment when they entered the greenhouse and when they left the greenhouse.

All hives entered the greenhouses with 22 frames of bees. Hives in the greenhouses with Zucchini declined very fast to approx. half the size if the colonies had received supplementary feed and to ¼ the size if they had not received supplementary feed.

Hives on the crops from the cabbage family did not decline in number of frames of bees if fed with the diet while they declined by ⅓ when not fed with the diet.

In case of Zucchini, the frames of brood declined in fed hives to 6/15^{th} of the number of frames present when they entered the greenhouse, compared to 3/15′ of the number of frames present when they entered the greenhouse, in the case of unfed hives (Table 6). In the cabbage crops the amount of brood did not decline in case of fed colonies while it halved in case of unfed colonies (Table 6).

Bee colonies fed according to the invention showed a bee bearding behavior (bees assembling outside the hive). Unfed colonies did not exhibit this phenomenon. This is an additional indication of increased bee activity and colony health in the fed colonies.

Importantly, colonies that were fed according to the invention recovered within 1 months to normal size (22 frames). Unfed colonies to recover from the stressful greenhouse pollination deployment only after two months.

Both fed and unfed colonies exhibit paralytic phenotypes probably caused by virus infections shortly after being placed in the greenhouses. However, after removal from the greenhouses, the fed colonies recovered from this paralytic phenotype within 1 brood cycle. The unfed colonies took 2-3 times as long to recover.

The data clearly shows that supplementary feeding a complete diet prior and/or during pollination of crops in greenhouses increases colony fitness as measured by colony size, brood production, activity and recovery rate from infection.

### BIBLIOGRAPHY

1. Pollen sterols are associated with phylogeny and environment but not with pollinator guilds. Zu, P, et al. 3, 2021, New Phytologist, Vol. 230, pp. 1169-1184.
2. Evaluating effects of a critical micronutrient (24-methylenecholesterol) on honey bee physiology. Chakrabarti, P, Lucas, H M and Sagili, R R. 3, 2020, Annals of the Entomological Society of America, Vol. 113, pp. 176-182.3. Nutritional physiology and ecology of honey bees. Wright, G A, Nicolson, S W and Shafir, S. 2018, Annual Review of Entomology, Vol. 63, pp. 327-344.
4. Honey bee health in Maine wild blueberry production. Drummond, F A, Lund, J and Eitzer, B. 6, 2021, Insects, Vol. 12, p. 523.
5. Diversity and nutritional value of pollen harvested by honey bee (Hymenoptera: Apidae) colonies during lowbush blueberry and cranberry (Ericaceae) pollination. Dufour, C, Fournier, V and Giovenazzo, P. 5, 2020, The Canadian Entomologist, Vol. 152, pp. 622-645.
6. Chemical composition of the “low quality” pollen of sunflower (Helianthus annuus, Asteraceae). Nicolson, S W and Human, H. 2013, Apidologie, Vol. 44, pp. 144-152.
7. The longevity of worker honey bees (Apis mellifera). Free, J B and Spencer-Booth, Y. 10-12, 1959, Proc R Entomol Soc, Vol. 34, pp. 141-150.
8. Seasonal change of the honeybee worker longevity in Sapporo, north Japan, with notes on some factors affecting lifespan. Fukuda, H and Sekiguchi, K. 5, 1966, Jpn J Ecol, Vol. 16, pp. 206-212.
9. Evolution and mechanisms of long life and high fertility in queen honey bees. Remonila, S C and Hughes, K A. 2008, AGE, Vol. 30, pp. 177-185.
10. Nutrition and health in honey bees. Brodschneider, R and Crailsheim, K. 3, 2010, Apidologie, Vol. 41, pp. 278-294.
11. Honey bee nutrition. Tsuruda, J M, Chakrabarti, P and Sagili, R R. 3, 2021, Veterinary Clinics of North America: Food animal practice, Vol. 37, pp. 505-519.
12. R Core Team. R: A language and environment for statistical computing. [Online]2023. https://ww.R-project.org.
13. Fitting linear mixed-effects models using lme4. Bates, D, et al. 1, 2015, Journal of Statistical Software, Vol. 67, p. 48.
14. Long-term global trends in crop yield and production reveal no current pollination shortage but increasing pollinator dependency. Aizen, M A, et al. 20, 2008, Current Biology, Vol. 18, pp. 1572-1575.
15. Importance of pollinators in changing landscapes for world crops. Klein, A M, et al.
2007, Proc. R. Soc. B., Vol. 274, pp. 303-313.
16. Potts, S G, Imperatriz-Fonseca, V L and Ngo, H T. The assessment report of the Intergovernmental Science-Policy Platform on Biodiversity and Ecosystem Services on pollinators, pollination and food production. s.l.: IPBES, 2016. ISBN 978-92-807-3567-3.
17. The economics of honey bee (Hymenoptera: Apidae) management and over wintering strategies for colonies used to pollinate almonds. DeGrandi-Hoffman, G, et al. 6, Journal of Economic Entomology, Vol. 112, pp. 2524-2533.
18. Worker nutrition and division of labour in honeybees. Toth, A L and Robinson, G E. 427-435, 2005, Anim. Behav., Vol. 69.
19. Nutritional regulation of division of labor in honey bees: Towards a systems biology perspective. Ament, S A, Wang, Y and Robinson, G E. 5, 2010, Wiley Interdiscip. Rev. Syst. Biol. Med., Vol. 2, pp. 566-576.
20. Influence of pollen nutrition on honeybee health: Dopollen quality and diversity matter. DiPasquale, G, et al. 8, 2013, Plos One, Vol. 8, p. e72016.
21. Diet effects on honeybee immunocompetence. Alaux, C, et al. 4, 2010, Biology letters, Vol. 6, pp. 562-565.
22. Honey constituent sup-regulate detoxification and immunity genes in the western honeybee Apis mellifera. Mao, W, Schuler, M A and Berenbaum, M R. 22, 2013, Proceedings of the National Academy of Sciences, Vol. 110, pp. 8842-8846.
23. Migratory management and environmental conditions affect lifespan and oxidative stress in honey bees. Simone-Finstrom, M, et al. 2016, Sci. Rep, Vol. 6, p. 32023.
24. Honey bee colonies provided with natural forage have lower pathogen loads and higher over winter survival than those fed protein supplements. DeGrandi-Hoffman, G, et al. 2015, Apidologie, Vol. 47, pp. 186-196.
25. Bond, J K, et al. Honey bees on the move: From pollination to honey production and back. ERR-290. Economic Research Service, US Department of Agriculture. 2021.
26. The effects of pollen availability during larval development on the behaviour and physiology of spring-reared honeybee workers. Mattila, H R and Otis, G W. 5, 2006, Apidologie, Vol. 37, pp. 533-546.
27. Drivers of colony losses. Steinhauer, N, et al. 2018, Curr Opin Insect Sci., Vol. 26, pp. 142-148.
28. Inner nest homeostasis in a changing environment with special emphasis on honeybee brood nursing and pollen supply. Schmickl, T and Crailsheim, K. 3, 2004, Apidologie, Vol. 35, pp. 249-263.
29. Honeybee colony loss linked to parasites, pesticides and extreme weather across the United States. Insolia, L, et al. 1, 2022, Scientific reports, Vol. 12, p. 20787.
30. Evaluating the strength of western honeybee (Apismellifera L.) colonies fed pollen substitutes over winter. Noordyke, E R, van Santen, E and Ellis, J D. 3, 2021, Journal of Applied Entomology, Vol. 146, pp. 291-300.
31. Pollen as food for bees: Diversity, nutrition, and contamination. Stoner, K A, Hendriksma, H P and Tosi, S. 2023, Frontiers in Sustainable Food Systems, Vol. 6, p. 1129358.
32. Honey bee (Apis mellifera) colony strength and its effects on pollination and yield in high bush blueberries (Vaccinium corymbosum). Grant, K J, DeVetter, L and Melathopoulos, A. Peer J, Vol. 9, p.e11634.
33. Appetite behavior of the honey bee Apis mellifera in response to phenolic compounds naturally found in nectars. Hernandez, I G, et al. 2, 2019, Journal of Experimental Biology, Vol. 222, p.jeb189910.
34. Interactions between pesticides and pathogen susceptibility in honey bees. O'Neal, S T, Anderson, T D and Wu-Smart, J Y. 2018, Current opinion in insect science, Vol. 26, pp. 57-62.
35. Bee declines driven by combined stress from parasites, pesticides, and lack of flowers. Goulson, D, et al. 6229, 2015, Science, Vol. 347, p. 1255957.
36. A systems-base approach to the environmental risk assessment of multiple stressors in honeybees. EFSA Scientific committee. 5, 2021, Efsa Journal, Vol. 19, p. e06607.
37. Are fungicides a driver of European foulbrood disease in honey bee colonies pollinating blueberries? Thebeau, J M, et al. 2023, Frontiers in Ecology and Evolution, Vol. 11, p. 1073775.
38. Are all colonies created equal? The role of honey bee colony strength in almond pollination contracts. Goodrich, B K and Goodhue, R E. 2020, Ecological Economics, Vol. 177, p. 106744.

What is claimed is:

1. A method for increasing pollination, crop quality and/or crop yield and/or seed production in agricultural or horticultural crops susceptible of pollination through invertebrates of the Apidae families (bees), in particular honey bees (Apini), bumble bees (Bombini), or stingless honey bees (Meliponini), comprising:
providing agricultural, horticultural crops, seed production crops or bee-yards where bees are placed in transit prior to or after pollination;
providing one or more beehives within or in the immediate vicinity of the agricultural or horticultural crops to pollinate the agricultural or horticultural crops during the pollination period of the agricultural or horticultural crops or greenhouse crops or partially covered crops, in particular tunnel crops;
administering to the beehives a pollen supplement composition
2-3 months prior to entry and at entry
during a part or the full pollination period of the agricultural or horticultural crops; and/or
during transportation from one crop to another crop; and/or
in the holding yards;
wherein the pollen supplement composition compensates for one or more dietary deficits in the amount or quality of the pollen of the agricultural or horticultural crops; and/or in the environment;
wherein the pollen supplement composition is free of pollen or comprises minor amounts of pollen, preferably less than 15 w % of pollen, even more preferably less than 10 w % of pollen, even more preferably less than 5 w % of pollen, even more preferably less than 1 w % of pollen;
optionally wherein the pollen supplement composition comprises one or more of:
fatty acids and omega-3 fatty acids is 5:1 to 1:20, preferably from 5:1 to 1:10, even more preferably from 2:1 to 1:5 (omega-6: omega-3) and wherein the omega-6 fatty acid preferably is linoleic acid and/or wherein the omega-3 fatty acid preferably is α-linolenic acid; and/or
sterols in an amount from 0.01 w % to 4 w % wherein the sterols preferably are one or more of cholesterol, 24-methylene cholesterol, fucosterol, isofucosterol, campesterol, beta-sitosterol and stigmasterol; and even more preferably comprise all aforementioned sterols; and/or
proanthocyanidins (multimers and/or monomers or their glucosides) in amount from 0.001 w % to 1 w %;
and wherein the w % is as compared to the total dry weight of the pollen supplement composition.

2. A method for increasing bee performance, comprising:
providing a pollen supplement composition; and
administering a pollen supplement composition to invertebrates of the Apidae families (bees), in particular honey bees (Apini), bumble bees (Bombini), or stingless honey bees (Meliponini);
wherein the pollen supplement composition is administered during the pollination period to bees deployed for pollination of the agricultural or horticultural crops susceptible of pollination by bees;
wherein the pollen supplement composition compensates one or more dietary deficits in the pollen of the agricultural or horticultural crops; and
optionally wherein the pollen supplement composition comprises one or more of:
fatty acids and omega-3 fatty acids is 5:1 to 1:20, preferably from 5:1 to 1:10, even more preferably from 2:1 to 1:5 (omega-6: omega-3) and wherein the omega-6 fatty acid preferably is linoleic acid and/or wherein the omega-3 fatty acid preferably is α-linolenic acid;
sterols in an amount from 0.01 w % to 4 w % wherein the sterols preferably are one or more of cholesterol, 24-methylene cholesterol, fucosterol, isofucosterol, campesterol, beta-sitosterol and stigmasterol; and even more preferably comprise all aforementioned sterols; and/or
proanthocyanidins in amount from 0.001 w % to 1 w %;
wherein the pollen supplement composition is free of pollen or comprises minor amounts of pollen as an appetizer, preferably less than 15 w % of pollen, even more preferably less than 10 w % of pollen, even more preferably less than 5 w % of pollen,
and wherein the w % is as compared to the total dry weight of the pollen supplement composition providing agricultural or horticultural crops.

3. A method for recovering performance of bees after the deployment for pollination of agricultural or horticultural crops susceptible of pollination by invertebrates of the Apidae families (bees), in particular honey bees (Apini), bumble bees (Bombini), or stingless honey bees (Meliponini), such as in a holding yard, comprising:
providing agricultural or horticultural crops;
providing one or more beehives within or in the immediate vicinity of the agricultural or horticultural crops to pollinate the agricultural or horticultural crops during the pollination period of the agricultural or horticultural crops or greenhouse crops or partially covered crops, in particular tunnel crops;
administering to the beehives a pollen supplement composition
Optionally during a part or the full pollination period of the agricultural or horticultural crops; and/or
During transportation from one crop to another crop; and/or
in the holding yards;
wherein the pollen supplement composition compensates for one or more dietary deficits in the amount or quality of the pollen of the agricultural or horticultural crops; and/or in the environment;
wherein the pollen supplement composition is free of pollen or comprises minor amounts of pollen, preferably less than 15 w % of pollen, even more preferably less than 10 w % of pollen, even more preferably less than 5 w % of pollen, even more preferably less than 1 w % of pollen; optionally wherein the pollen supplement composition comprises one or more of:
fatty acids comprising omega-6 and omega-3 fatty acids wherein the ratio of omega-6 fatty acids and omega-3 fatty acids is 5:1 to 1:20, preferably from 5:1 to 1:10, even more preferably from 2:1 to 1:5 (omega-6: omega-3) and wherein the omega-6 fatty acid preferably is linoleic acid and/or wherein the omega-3 fatty acid preferably is α-linolenic acid; and/or
sterols in an amount from 0.01 w % to 4 w % wherein the sterols preferably are one or more of cholesterol, 24-methylene cholesterol, fucosterol, isofucosterol, campesterol, beta-sitosterol and stigmasterol; and even more preferably comprise all aforementioned sterols; and/or
proanthocyanidins (multimers and/or monomers or their glucosides) in amount from 0.001 w % to 1 w %;
and wherein the w % is as compared to the total dry weight of the pollen supplement composition.

4. A method for preparing bees for deployment in pollination of agricultural or horticultural crops susceptible of pollination by invertebrates of the Apidae families (bees), in particular honey bees (Apini), bumble bees (Bombini), or stingless honey bees (Meliponini), such as in a holding yard, comprising:
providing a pollen supplement composition; and
administering a pollen supplement composition to bees in advance of their deployment for pollination of agricultural or horticultural crops susceptible of pollination by bees;
providing agricultural or horticultural crops;
providing one or more beehives within or in the immediate vicinity of the agricultural or horticultural crops to pollinate the agricultural or horticultural crops during the pollination period of the agricultural or horticultural crops or greenhouse crops or partially covered crops, in particular tunnel crops;
administering to the beehives a pollen supplement composition
during a part or the full pollination period of the agricultural or horticultural crops; and/or
during transportation from one crop to another crop; and/or
in the holding yards;
wherein the pollen supplement composition compensates for one or more dietary deficits in the amount or quality of the pollen of the agricultural or horticultural crops; and/or in the environment;
wherein the pollen supplement composition is free of pollen or comprises minor amounts of pollen, preferably less than 15 w % of pollen, even more preferably less than 10 w % of pollen, even more preferably less than 5 w % of pollen, even more preferably less than 1 w % of pollen; optionally wherein the pollen supplement composition comprises one or more of:
fatty acids and omega-3 fatty acids is 5:1 to 1:20, preferably from 5:1 to 1:10, even more preferably from 2:1 to 1:5 (omega-6: omega-3) and wherein the omega-6 fatty acid preferably is linoleic acid and/or wherein the omega-3 fatty acid preferably is α-linolenic acid; and/or
sterols in an amount from 0.01 w % to 4 w % wherein the sterols preferably are one or more of cholesterol, 24-methylene cholesterol, fucosterol, isofucosterol, campesterol, beta-sitosterol and stigmasterol; and even more preferably comprise all aforementioned sterols; and/or
proanthocyanidins (multimers and/or monomers or their glucosides) in amount from 0.001 w % to 1 w %;
and wherein the w % is as compared to the total dry weight of the pollen supplement composition.

5. The method according to claim 1, wherein the source of the proanthocyanidins is a non-pollen tissue of one or more of Solanaceae, Poaceae, Ranunculaceae, Fabaceae, Corylaceae, Cranberry/Blueberry; Apple (Rosaceae), Vitaceae.

6. The method according to claim 1, wherein the pollen supplement composition further comprises amino acids in an amount of 10 w % to 30 w %, preferably 15 w % to 25 w %, wherein the amount of essential amino acids is
10 w % to 15 w % arginine;
3 w % to 8 w % histidine;
1 w % to 5 w % isoleucine;
8 w % to 13 w % leucine;
40 w % to 50 w % lysine;
2 w % to 7 w % methionine;
3 w % to 8 w % phenylalanine;
4 w % to 9 w % threonine;
5 w % to 15 w % valine; and/or
5 w % to 10 w % tryptophan
as compared to the total of essential amino acids in the pollen supplement composition.

7. The method according to claim 1, wherein the pollen supplement composition further comprises one or more of
vitamins, preferably one or more of B1, B2, B3, B4, B5, B9, B7, B12, Inositol, A, C, E, Choline, preferably in a concentration of 0.0001 w % to 2 w %, preferably from 0.02 w % to 1 w %;
minerals, preferably one or more of zinc, iron, manganese, magnesium, copper, potassium, phosphorous, calcium and sodium;
wherein the w % is as compared to the total dry weight of the pollen supplement composition.

8. The method according to claim 1, wherein the background of the pollen supplement composition is as follows:
protein: 10 w % to 30 w %, preferably 15 w % to 25 w %,
carbohydrates, preferably sugars, even more preferably glucose, fructose or a mixture thereof: 0 w % to 70 w %, preferably 15 w % to 60 w %,
lipids comprising the sterols and fatty acids: 1 w % to 15 w %, preferably 5 w % to 10 w %;
wherein the w % is as compared to the total dry weight of the pollen supplement composition.

9. The method according to claim 1, wherein the pollen supplement composition is as follows:
dry matter: 60 w % to 90 w %;
ash: 0.5 w % to 5 w %, preferably 1 w % to 4 w %; and
fiber: 0.1 w % to 5 w %, preferably 0.5 w % to 1 w %,
wherein the w % is as compared to the total dry weight of the pollen supplement composition.

10. The method according to claim 1, wherein the dry matter of the pollen supplement composition is 70 w % or more, preferably 75 w % or more, even more preferably 80 w % or more, wherein the w % is as compared to the total dry weight of the pollen supplement composition.

11. The method according to claim 1, wherein the crop is a monoculture and wherein monoculture means a field cultivated with plants of one genus or one species of at least 0.2 ha, preferably at least 1 ha, preferably at least 5 ha, even more preferably at least 10 ha, even more preferably at least 50 ha, even more preferably at least 100 ha or in case of seed production an insect screened enclosure.

12. The method according to claim 1, wherein the agricultural crop is of one or more of the following families:
Ericaceae, preferably blueberry (Vaccinium sect. Cyanococcus), or cranberry (Vaccinium subg. Oxycoccus)
Rosaceae, preferably almond (Prunus amygdalus), Apple (Malus domestica), cherry (Prunus avium), pear (Pyrus communis), raspberry (Rubus idaeus), blackberry (Rubus subg. Rubus)
Asteraceae, preferably sunflower (Helianthus annuus),
Brassicaceae, preferably canola (Brassica napus), Rapeseed (Brassica napus subsp napus), Cabbage (red, green, broccoli, cauliflower, sprouts), (Brassica oleracea)
Lauraceae: Avocado
Cucurbitaceae, preferably cucumber (Cucumis sativus L.), melon (Cucumis melo L.), watermelon (Citrullus lanatus), calabash (Lagenaria siceraria), or pumpkin (Cucurbita spp.)
Umbellifers;
Apiaceae, preferably carrot (Daucus carota) or fennel (Foeniculum vulgare);
Amaryllidaceae, preferably onion (Allium cepa L.)
Rutaceae, preferably citrus (Citrus L.), or
Fabaceae, preferably alphalpha (Medicago sativa), soy (Glycine max), or peas (Lathyrus oleraceus),
wherein the horticultural crop is of one or more of the following families:
Cucurbitaceae, preferably zucchini (Cucurbita pepo), or
Solanaceae, preferably Aubergine (Solanum melongena).

13. The method according to claim 1, wherein the pollen supplement composition is free of or does only contain minor amounts, such as less than 5 w %, preferably less than 1 w %, even more preferably less than 0.1 w % of one or more of bee pheromones, sabinene, beta-pinene, limonene, nicotine, caffeine, floral fragrance components, isophorone and 4-oxoisophorone, citral, benzaldehyde, linalool, alpha-pinene, cinnamyl alcohol, and cis-3-hexenyl acetate.

14. The method according to claim 1, wherein the pollen supplement composition is administered
in solid form such as a patty or in liquid form such as a solution or spray;
inside or outside the beehive.

15. The method according to claim 1, wherein the increased bee performance and/or the increased pollination, crop quality and/or crop yield are obtained by one or more of:
Reduced colony mortality;
Increased number of bees produced;
Increased number of capped brood;
Increased bee population size;
Improved bee fitness and vitality;
Increased pollination performance;
Increased fruit quality, preferably size, shape or form;
Increased fruit quantity, and
Reduced rejection rate of fruit
Increased vegetable quantity or fruit quality, preferably size, shape or form,
Increased vegetable quantity or fruit quantity,
Reduced rejection rate of vegetables fruit, and
Increased seed quantity.
